(19) United States
(12) Patent Application Publication
(10) Pub. No.: US 2025/0312383 A1
(43) Pub. Date: Oct. 9, 2025

(54) HUMAN UMBILICAL CORD BLOOD FOR USE IN ALLEVIATING FATIGUE

(71) Applicant: STEMCYTE, INC., Baldwin Park, CA (US)

(72) Inventors: Tong-Young LEE, Taipei City (TW); Yu-Chin SU, Taipei City (TW); Ying-Chieh CHEN, Taipei City (TW); Chih-Hsin CHEN, Taipei City (TW)

(21) Appl. No.: 18/627,750

(22) Filed: Apr. 5, 2024

(51) Int. Cl. A61K 35/51; A61K 9/00; A61K 47/20; A61K 47/36; A61P 43/00; G01N 33/68

(52) U.S. Cl. CPC A61K 35/51; A61K 9/0026; A61K 47/20; A61K 47/36; A61P 43/00; G01N 33/6863; G01N 33/6893; G01N 2800/52

(57) ABSTRACT

A method for alleviating fatigue, comprising administering to a patient in need thereof an effective amount of a human umbilical cord blood composition, which comprises hematopoietic progenitor cells, monocytes, lymphocytes, granulocytes, or a combination thereof.

### BACKGROUND OF THE INVENTION

Fatigue is a feeling of extreme tiredness, weariness, lack of energy, or a combination thereof. It can interfere with a person's normal daily activities. A person with fatigue may feel weak, worn out, unable to concentrate, and/or suffer from short-term memory loss or emotional changes. Figure can be caused by various factors such as medical conditions, illness, unhealthy lifestyle choices, grief and stress.

Coronavirus disease of 2019 (COVID-19) has resulted in substantial mortality and morbidity worldwide. Up to one-third people recovered from COVID-19 still suffer from post-COVID syndrome (a.k.a., long COVID), including cognitive dysfunction and fatigue. As a result, nearly a quarter of post-COVID patients could not return to work.

Accordingly, it is of great interest to develop effective approaches to alleviate fatigue, including fatigue associated with post-COVID syndrome.

### SUMMARY OF THE INVENTION

The present disclosure is based, at least in part, on the superior efficacy of human umbilical cord blood in alleviating fatigue in post-COVID patients as observed in a Phase IIa clinical trial.

Accordingly, provided herein, in some aspects, is a method for alleviating fatigue, comprising administering to a subject in need thereof an effective amount of a human umbilical cord blood (hUCB) composition. The hUCB composition comprises cells such as hematopoietic progenitor cells, monocytes, lymphocytes, granulocytes, or a combination thereof. In some embodiments, the subject is a human patient suffering from fatigue. For example, the human patient has post-COVID syndrome or chronic fatigue syndrome. Any of the subject for treatment by the method disclosed herein may have SARS-CoV-2 infection at least 6 months prior to the first dose of the human umbilical cord blood composition with post-COVID syndrome.

In some embodiments, the hUCB composition disclosed herein may comprise comprises 5-10% dimethyl sulfoxide (DMSO) by volume and 0.5-1% dextran by volume, in which the cells suspend. The hUCB composition may be cryopreserved prior to administration. In some embodiments, the subject receives one or more doses of the human umbilical cord blood composition in the treatment method. In some instances, each dose is about 1×10⁷ to about 9.0×10⁷ total nucleated cells. In some examples, each dose is about 1×10⁷ to about 8.6×10⁷ in the human umbilical cord blood composition per kilogram of the subject. In one specific example, each dose is about 1×10⁷ total nucleated cells in the human umbilical blood composition per kilogram of the subject. In some examples, each dose of the human umbilical cord blood composition has a volume of 25 mL, which is deemed a dosage form of the hUCB composition).

In some embodiments, the method disclosed herein comprises three doses of the human umbilical cord blood composition, each of which is about 1×10⁷ to about 9.0×10⁷ total nucleated cells per kilogram of the subject (e.g., about 1×10⁷ to about 8.6×10⁷ in the human umbilical cord blood composition per kilogram of the subject). Among the three doses, two consecutive doses are three weeks ±3 days apart. In some examples, one or more of the three doses of the hUCB composition contain about 1×10⁷ total nucleated cells per kilogram of the subject; and wherein two consecutive doses of the three doses are three weeks ±3 days apart.

In some embodiments, the human umbilical cord blood composition is allogenic to the subject. Alternatively or in addition, the ABO/Rh type of blood cells in the human umbilical cord blood composition matches that of the subject.

In some embodiments, the human umbilical cord blood composition is administered to the subject by intravenous infusion. When the subject is a human adult patient, the infusion rate may be 0.5-5 mL per minute. In some examples, the infusion rate for a human adult patient may be 1.5-2 mL per minute.

Any of the methods disclosed herein may further comprise monitoring occurrence of adverse effects after administration of the human umbilical cord blood composition. The dose of the hUCB composition may be reduced or the treatment may be discontinued when a severe adverse effect occurs. Alternatively or in addition, the method disclosed herein may further comprise assessing treatment efficacy. Examples include assessing levels of fatigue, cognition, life quality, or a combination thereof via CFQ-11 scale assessment, PGI-S scale assessment, FRAIL scale assessment, MoCA scall assessment, EQ-5D-5L scale assessment, or a combination thereof. In some embodiments, the method may further comprise measuring levels of biomarkers associated with frailty, cognition, and/or fatigue (e.g., Cystatin C, insulin-like growth factor 1 (IGF-1), and/or ehydroepiandrosterone sulfate (DHEA-S)), and/or levels of inflammatory cytokines (e.g., cutaneous T cell-attracting chemokine (CTACK), C-X-C motif chemokine ligand 10 (IP-10), interleukin 18 (IL-18), neural cell adhesion molecule 1 (NCAM-1), kallikrein-related peptidase 6 (KLK-6), and chitinase-3 like protein 1 (CHI3L1)) in in one or more blood samples of the subject. In some instances, the blood samples are collected before, during, and/or after administration of the human umbilical blood cord composition.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic illustrating depicting study schema of a randomized, controlled Phase IIa clinical study with a human umbilical cord blood composition in human patients with post-COVID syndrome. EOS: end of study. EOT: end of treatment. IP: investigational product (human umbilical cord blood composition). R: randomization. W: week.

FIGS. 2A-2B include diagrams showing change of CFQ-11 biomodal total scores in patients treated with hUCB or placebo. FIG. 2A: CFQ-11 biomodal score chart. FIG. 2B: CFQ-11 biomodal score chart showing changes from baseline. *p<0.05, ***p<0.001.

FIGS. 3A-3B: include diagrams showing change of CFQ-11 Likert fatigue scale in patients treated with hUCB or placebo. FIG. 3A: CFQ-11 Likert score chart. FIG. 3B: CFQ-11 Likert score chart showing changes from baseline. *p<0.05, **p<0.01, ***p<0.001.

FIGS. 4A-4B: include diagrams showing change of physical fatigue shown by CFQ-11 fatigue scale in patients treated with hUCB or placebo. FIG. 4A: CFQ-11 physical fatigue score chart. FIG. 4B: CFQ-11 physical fatigue score chart showing changes from baseline. *p<0.05, **p<0.01, ***p<0.001.

FIGS. 5A-5B: include diagrams showing FRAIL scale changes in patients treated with hUCB relative to patients treated with the placebo. FIG. 5A: FRAIL scores at various time points after treatment. FIG. 5B: FRAIL score changes from baseline.

FIGS. 6A-6L: include diagrams showing EQ-5D-5L score changes in patients treated with hUCB relative to patients treated with the placebo. FIGS. 6A-6B: EQ-5D-5L score changes for mobility (6A) and score changes from baseline (6B). FIGS. 6C-6D: EQ-5D-5L score changes for self-care (6C) and score changes from baseline (6D). FIGS. 6E-6F: EQ-5D-5L score changes for usual activities (6E) and score changes from baseline (6F). FIGS. 6G-6H: EQ-5D-5L score changes for pain and discomfort (6G) and score changes from baseline (6H).

FIGS. 6I-6J: EQ-5D-5L score changes for anxiety and depression (6I) and score changes from baseline (6J). FIGS. 6K-6L: EQ-5D-5L score changes for health (6K) and score changes from baseline (6L). *p<0.05.

FIGS. 7A-7B: include diagrams showing MoCA scale changes in patients treated with hUCB relative to patients treated with the placebo. FIG. 7A: MoCA stores at various time points after treatment. FIG. 7B: MoCA score changes from baseline.

FIGS. 8A-8C include charts showing changes of biomarkers associated with fatigue, cognition, and frailty in patients treated with hUCB as compared with patients treated with the placebo. FIG. 8A: Cystatin C. FIG. 8B: IGF-1. FIG. 8C: DHEA-S. *p<0.05.

FIGS. 9A-9F include charts showing changes of inflammatory cytokine levels in patients treated with hUCB as compared with patients treated with the placebo. FIG. 9A: CTACK. FIG. 9B: IL-10. FIG. 9C: IL-18. FIG. 9D: NCAM-1. FIG. 9E: Kallikrein-6 (KLK-6). FIG. 9F: CHI3L1/YKL-40. *p<0.05, and **p<0.01.

### DETAILED DESCRIPTION OF THE INVENTION

Reported herein is a randomized controlled Phase IIa clinical trial to assess safety and efficacy of human umbilical cord blood (hUCB) infusion in patients with post-COVID syndrome, e.g., fatigue. Results from this Phase IIa showed superior efficacy of the hUCB composition in alleviating fatigue, improving cognition, and improving life quality, indicating that hUCB would be a promising therapeutic agent for such purposes in patients who need the treatment, for example, patients suffering from fatigue such as fatigue associated with post-COVID. Accordingly, provided herein are methods for alleviating fatigue using hUCB compositions.

### I. Human Umbilical Cord Blood Compositions

The umbilical cord connects a baby to the placenta of the mother, which grows in the uterus and supplies the baby with nutrients and oxygen through the umbilical cord. Cord blood is the blood from a baby that is left in the umbilical cord and placenta after birth. Umbilical cord blood contains hematopoietic stem cells, which can grow into different types of blood cells. Human umbilical cord blood has been used for treating various diseases.

The human umbilical cord blood composition disclosed herein contain human umbilical cord blood (hUCB), which can be collected by drawing the blood from the umbilical vessels and placenta into a collection bag after a baby is born. The hUCB composition comprises stem cells and progenitor cells (e.g., CD34+ hematopoietic stem cells), and mononuclear cells, for example, monocytes, lymphocytes, granulocytes, or a combination thereof. The cellular composition of each hUCB composition would depend on the composition of cells in the blood recovered from the umbilical cord and placenta of the donor.

The collected hUCB may be suspended in a cryopreservation solution comprising one or more suitable excipients to preserve viability and bioactivity of the cells and optionally additional active agents in the hUCB. In some embodiments, the cryopreservation solution for use in the present disclosure may comprise adenosine, dextrose, dextran-40, lactobionic acid, sucrose, mannitol, a buffer agent such as N-)2-hydroxyethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some examples, the hUCB composition provided herein may comprise hUCB suspended in dimethyl sulfoxide (DMSO) and dextran at suitable concentrations, for example, about 5-10% DMSO and about 0.5-1% dextran (e.g., Dextran 40). In one example, the hUCB composition comprises hUCB, 10% DMSO and 1% Dextran 40.

The hUCB compositions may be analyzed to determine potential potency and safety features, for example, nucleated cell count, cell viability, count of CD34⁺ cells, potency, identity, and colony forming unit assays. The potency of cord blood is determined by measuring the numbers of total nucleated cells (TNC) and CD34⁺ cells, and cell viability. In some instances, the ABO group and HLA typing can be determined for each hUCB composition.

In some instances, the hUCB composition provided herein can be in dosage form, which refers to the form that would be administered to a subject. For example, the dosage form of the hUCB provided herein may comprise a suitable amount of total nucleated cells (TNC), which may range from around 5×10⁸ TNC to 4.3×10⁹ TNC suspended in 10% DMSO and 1% Dextran 40. In some examples, the dosage form of the hUCB composition provided herein may comprise about 5×10⁸ TNC suspended in 10% DMSO and 1% Dextran 40. In some examples, the dosage form of the hUCB composition provided herein may comprise about 6×10⁸ TNC suspended in 10% DMSO and 1% Dextran 40. In some examples, the dosage form of the hUCB composition provided herein may comprise about 7×10⁸ TNC suspended in 10% DMSO and 1% Dextran 40.

In some examples, the dosage form of the hUCB composition provided herein may comprise about 8×10⁸ TNC suspended in 10% DMSO and 1% Dextran 40. In some examples, the dosage form of the hUCB composition provided herein may comprise about 9×10⁸ TNC suspended in 10% DMSO and 1% Dextran 40. In some examples, the dosage form of the hUCB composition provided herein may comprise about 1×10⁹ TNC suspended in 10% DMSO and 1% Dextran 40.

In some examples, the dosage form of the hUCB composition provided herein may comprise about 2×10⁹ TNC suspended in 10% DMSO and 1% Dextran 40. In some examples, the dosage form of the hUCB composition provided herein may comprise about 3×10⁹ TNC suspended in 10% DMSO and 1% Dextran 40. In some examples, the dosage form of the hUCB composition provided herein may comprise about 4×10⁹ TNC suspended in 10% DMSO and 1% Dextran 40. Any of the dosage forms disclosed herein may be in a total volume of 25 mL.

Any of the hUCB can be cryopreserved and stored under suitable conditions. Prior to use, the frozen hUCB can be thawed on site under suitable conditions, e.g., at 37° C.

### II. Methods for Alleviating Fatigue

Any of the hUCB compositions provided herein can be used to alleviate fatigue, improve cognition, and/or improve life quality. Accordingly, the present disclosure provides a method of using the hUCB composition disclosed herein to achieve any of the intended outcome.

### (A) Subjects for Treatment

The method provided herein can be performed on subjects who suffer from fatigue. Fatigue refers to extreme tiredness resulting from mental or physical exertion or illness. In some embodiments, the subject for treatment is a human patient suffering from fatigue. In some examples, the human patient has chronic fatigue syndrome (also called myalgic encephalomyelitis). In other examples, the human patient may have post-viral infection fatigue, for example, post-COVID with fatigue. In yet other examples, the human patient may have fatigue associated with illness, for example, cancer or stroke. In still another example, the huma patient has fibromyalgia.

In some embodiments, the subject for treatment by any of the methods disclosed herein is a human patient having post-COVID. Coronavirus disease 2019 (COVID-19) is caused by severe acute respiratory syndrome coronavirus 2 (SARS-CoV-2), a newly discovered virus in the family of Coronaviridae (beta). Most people diagnosed with COVID-19 recover within weeks of illness, while some people experience post-COVID conditions that can last weeks or months after being infected with SARS-CoV-2. Post-COVID is also known as long COVID, long-haul COVID, post-acute COVID-19, long-term effects of COVID, or chronic COVID. Anyone who becomes ill with COVID-19 can develop post-COVID. In some instances, a patient with post-COVID exhibits signs and symptoms that develop during or after an infection with SARS-CoV-2 and continue for more than 6 months but not over 18 months, and are not explained by an alternative diagnosis. In some instances, a post-COVID patient has a history of probable or confirmed SARS-CoV-2 infection, usually within three months from the onset of COVID-19, with symptoms and effects that last for at least two months. The symptoms and effects of post-COVID condition cannot be explained by an alternative diagnosis. In some instances, patients with post-COVID may have a range of new or ongoing symptoms that can last weeks or months after they are infected with the virus that causes COVID-19 and that can worsen with physical or mental activity. In other instances, post-COVID patients may have a constellation of symptoms developed during or following COVID-19 infection, persist for >12 weeks, and are not sufficiently explained by alternative diagnoses.

Post-COVID can have a significant effect on people's quality of life and challenges for determining best-practice still present. The methods provided here can provide a reliable solution to solve this problem.

Common symptoms of post-COVID include extreme tiredness (fatigue), shortness of breath, chest pain or tightness, problems with memory and concentration (brain fog), difficulty sleeping (insomnia), heart palpitations, dizziness, pins and needles, joint pain, depression and anxiety, tinnitus or earaches, feeling sick, diarrhea, stomach aches, or loss of appetite, a high temperature, cough, headaches, sore throat, changes to sense of smell or taste, and/or rashes.

The subject for treatment in any of the methods disclosed herein may be a human patient, who may be aged ≥18 and ≤65 years. In some embodiments, the human patient exhibits post-COVID syndrome, e.g., signs and symptoms that develop during or after an infection consistent with COVID-19, continue for more than 6 months but not over 18 months, and are not explained by an alternative diagnosis. Such a human patient may be confirmed with novel coronavirus (SARS-CoV-2) infection as determined by COVID-19 rapid antigen test or SARS-CoV-2 polymerase chain reaction (PCR) test at least 6 months prior to screening visit. In some instances, the human patient may have a recent (e.g., within 7 days) negative SARS-CoV-2 test (e.g., an approved PCR or antigen test).

In other embodiments, the subject is a human patient suffering from fatigue caused by factors not related to COVID, e.g., mental or physical exertion or illness not related to COVID. Some examples are provided herein. In one specific example, the human patient may have chronic fatigue syndrome (CFS). CFS, also known as myalgic encephalomyelitis (ME), is a serious long-term illness that affects many body systems. CFS is common in people between 40-60 years old, although anyone can get CFS. The causes for CFS have yet identified. It is suggested that CFS can be triggered by a combination of multiple factors. Some examples are provided below: infections, immune system changes, stress affecting body chemistry, changes in energy production, and/or possible genetic link. Symptoms of CFS appear similar to many other illness, including greatly lowered ability to do activities that were usual before the illness, worsening of symptoms after physical or mental activity that would not have caused a problem before the illness, sleep problems, problems with thinking and memory, and/or worsening of symptoms while standing or sitting upright (orthostatic intolerance).

In some instances, the human patient may meet the criteria for fatigue, for example, based on the CFQ-11 case definition of fatigue as known in the art (see discussions below). In some instances, the human patient may have self-reported concerns regarding cognitive functioning or recent diagnosis of COVID-19 related cognitive impairment. Alternatively or in addition, the human patient may have a MoCA test score ≤28/30 prior to treatment. Further, the ABO/Rh blood type of the patient may match that of the hUCB composition.

Further, the patient may not have one or more of the following: (a) confirmed to have persistent fatigue prior to COVID; (b) has neurological disorders prior to COVID-19 diagnosis; (c) has been hospitalized in intensive care unit (ICU) within 1 year; (d) has Known hypersensitivity to dimethyl sulfoxide (DMSO) or Dextran-40_(e) had previously received stem cell therapy; (f) with known immune disease; (g) is pregnant or breastfeeding; and (h) has received any vaccination within 3 weeks prior to the first IP infusion.

Further, the human patient may not be under conditions that may increase risk of complications based on, for example, the parameters as below: blood pressure systolic >160 or <90 mmHg or diastolic >100 or <60 mmHg; pulse <60 or >105 bpm; Respiratory rate <9 and >28 bpm; pulse oximetry <94% in room air; body temperature >100.4 degrees Fahrenheit; alanine aminotransferase (ALT) or aspartate aminotransferase (AST) >2 times the upper limit of normal (ULN); abnormal bilirubin unless subject has Gilbert's syndrome; estimated glomerular filtration rate (eGFR)<60 mL/min/1.73 m² by Chronic Kidney Disease Epidemiology Collaboration (CKD-EPI); hemoglobin <10 mg/dL; and/or platelet count <100,000/μL. In some instances, the human patient may have no presence of one or more of the following: chronic heart failure (CHF) in New York Heart Association (NYHA) class II to IV; chronic kidney disease (CKD) in stage 3b to 5; acute respiratory distress syndrome (ARDS); psychiatric condition that is likely to interfere with the conduct of the study (e.g., chronic depression); and chronic pulmonary disease.

In some instances, the subject for treatment meets one or more of the inclusion and exclusion criteria listed in Example 1 below.

### (B) Treatment Conditions

To practice the method disclosure herein, an effective amount of the hUCB composition as disclosed herein can be administered to a subject in need of the treatment (e.g., the human patients described above) via a suitable delivery route. In some instances, the hUCB composition is delivered by intravenous infusion.

As used herein, the term “treating” as used herein refers to the application or administration of a composition including one or more active agents to a subject, who has fatigue or other conditions described herein, with the purpose to cure, heal, alleviate, relieve, alter, remedy, ameliorate, improve, or affect the disease, the symptoms of the disease, or the predisposition toward the disease.

Alleviating a target disease/disorder (e.g., fatigue) includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

The hUCB composition disclosed herein is administered to a subject in need of the treatment in an effective amount. An “effective amount” is that amount of the hUCB (determined based on the total nucleated cell number or TNC thereof) that alone, or together with further doses, produces the desired response, e.g., alleviating fatigue, improving cognition, and/or improving life quality. In the case of treating a particular disease or condition, the desired response is inhibiting the progression of the disease. This may involve only slowing the progression of the disease temporarily, although more preferably, it involves halting the progression of the disease permanently. This can be monitored by routine methods or can be monitored according to diagnostic methods such as those provided in Example 1 below. The desired response to treatment of the disease or condition also can be delaying the onset or even preventing the onset of the disease or condition.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In some embodiments, the subject for treatment (e.g., a human patient as described above) may be given the hUCB composition via intravenous infusion at a dose of about 1×10⁷ to about 9.0×10⁷ total nucleated cells in the human umbilical cord blood composition per kilogram of the subject. In some examples, the subject for treatment (e.g., a human patient as described above) may be given the hUCB composition via intravenous infusion at a dose of about 1×10⁷ to about 8.6×10⁷ total nucleated cells in the human umbilical cord blood composition per kilogram of the subject. In some specific examples, the dose of the hUCB composition may be about 1×10⁷ total nucleated cells in the hUCB composition per kilogram of the subject.

In some embodiments, multiple doses of the hUCB composition may be given to the human patient, e.g., via intravenous infusion. When the subject is a human adult patient, the infusion rate may range from about 0.5-5 mL per minute. In some examples, the infusion rate for a human adult patient may be about 1.5-2 mL per minute.

In some examples, a human patient is administered three doses of the hUCB composition. In some instances, the two consecutive doses may be about 3 wks ±3 days apart. In some instances, one or more of the multiple doses may range from about 1×10⁷ to about 9.0×10⁷ (e.g., 1×10⁷ to about 8.6×10⁷) about total nucleated cells in the human umbilical cord blood composition per kilogram of the subject. In some examples, the one or more doses may be about 1×10⁷ total nucleated cells in the hUCB composition per kilogram of the subject. In some instances, the human patient may be given three identical doses. In other instances, the human patient may be given three different doses, for example, a lower second or third dose relative to the first dose (e.g., when some adverse events were observed after the first dose).

In some embodiments, the hUCB composition can be formulated in a cryopreservation solution as disclosed herein (e.g., containing about 5-10% DMSO and about 0.5-1% Dextran 40). In some examples, the hUCB composition is in a dosage form, which may contain the required dosage of hUCB suspended in 10% DMSO and 1% Dextran 40 in a total volume of 25 mL. The hUCB composition may be cryopreserved and stored under suitable conditions to maintain cell viability and bioactivity and thawed prior to administration on site.

### (C) Post-Treatment Assessment

In some embodiments, a subject for treatment (e.g., a human patient as described herein) may subject to assessment of features relating to, for example, adverse events and/or treatment efficacy. For example, assessment regarding fatigue levels may be performed to the human patient after the treatment, e.g., at various time points after treatment. This may be performed using the CFQ-11 scales as detailed in Example 1 below, including, for example, CFQ-11 bimodal scale, CFQ-11 Likert scale, CFQ-11 physical fatigue scale, and/or CFQ-11 mental fatigue scale. In some instances, assessment based on PGI-S may be performed. Alternatively or in addition, assessment based on FRAIL scale may be performed. In some instances, assessment for quality of life, e.g., using the EQ-5D-5L scales, may be performed. In other instances, assessment for cognition, e.g., using the MoCA scales, may be performed. Results from the Phase IIa clinical trial disclosed herein indicate that the hUCB composition disclosed here is effective in alleviating fatigue, improving cognition, and improving quality of life. The assessment provided herein can be used to determine treatment efficacy and adjustment of treatment conditions accordingly.

In some embodiments, levels of certain biomarkers associated with fatigue, frailty, and/or cognition (e.g., Cystatin C, IGF-1, and DHEA-S) in blood samples obtained from a human after treatment may be measured to analyze impact of the hUCB composition on these features. Alternatively or in addition, levels of inflammatory cytokines in blood samples obtained from a human after treatment may be measured to assess impact of hUCB on such inflammatory cytokines. Examples include, but are not limited to, CTACK, IP-10, IL-18, NCAM-1, KLK-6, and CHI3L1/YKL-40. Results from the Phase IIa clinical trial disclosed herein indicate that the hUCB composition disclosed here modulates levels of these biomarkers and/or inflammatory cytokines in a direction of improving health conditions. The assessment provided herein can be used to determine treatment efficacy and adjustment of treatment conditions accordingly.

In some embodiments, the human patients are monitors for occurrence of adverse effects after treatment, for example, allergic reactions to the hUCB composition or components thereof. Management of such adverse effects is to be performed following routine practice when necessary. If severe adverse events are observed, doses of the hUCB composition may be reduced or treatment may be terminated.

### III. Kits for Use in Alleviating Fatigue

The present disclosure also provides kits for use of alleviating fatigue or other medical conditions as disclosed herein with the hUCB composition provided herein.

A kit for therapeutic use as described herein may include one or more containers comprising a hUCB composition disclosed herein, which may be formulated to form a pharmaceutical composition.

In some embodiments, the kit can additionally comprise instructions for use of the hUCB composition in any of the methods described herein. The included instructions may comprise a description of administration of the hUCB composition to a subject in need of the treatment to achieve the intended efficacy in the subject. The kit may further comprise a description of selecting a subject suitable for the treatment based on the inclusion and/or exclusion criteria provided in Example 1 above. In some embodiments, the instructions comprise a description of administering the hUCB composition to a subject who is in need of the treatment.

The instructions relating to the use of the hUCB composition as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (lRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or patient matter referenced herein.

### Example 1: A Randomized Controlled Phase IIa, Two-Arm Study to Assess the Safety and Efficacy of Human Umbilical Cord Blood Infusion in Patients with Post-COVID Syndrome

The influence of post-COVID syndrome is as severe as COVID-19, and its principal symptoms, including cognitive impairment and fatigue, have been producing global health and economic issues. Notably, small molecule drugs or biologics obviously treat post-COVID patients ineffectively. Crook et al., BMJ 2021; 374.

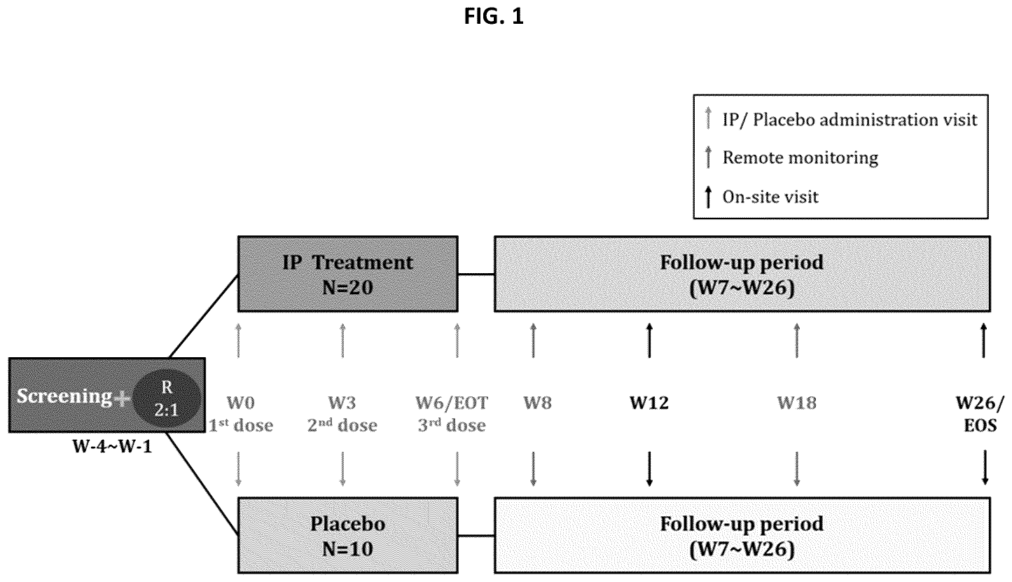

This example describes a randomized controlled Phase IIa study to assess safety, tolerability, and efficacy of a human umbilical cord blood composition in human patients with post-COVID syndrome such as fatigue. Post-COVID is defined as patients with signs and symptoms that develop during or after an infection consistent with COVID-19, continue for more than 6 months but not over 18 months, and are not explained by an alternative diagnosis. The purpose of this phase IIa study is to investigate the safety, feasibility, and efficacy of hUCB infusion in patients suffering from post-COVID fatigue and cognitive impairment. The study schema is illustrated in FIG. 1.

### I. Investigational Product

Human umbilical cord blood (hUCB), the investigational product of the Example, is collected from the placenta either ex utero or in utero at the time of delivery using a functionally closed collection system. The blood drains from the cord into the collection bag containing anticoagulant citrate-phosphate-dextrose (CPD). Collection volumes range from 60-200 mL. Informed consent is obtained from every donor mother and samples of the mother's blood are tested for relevant communicable disease agents or diseases (RCDAD) including, Hepatitis B, Hepatitis C, HIV-1/2, HTLV-I/II, and syphilis as well as for CMV. A maternal medical history is obtained, and a six-month follow-up of the baby is requested.

The collection bag, cryobag, test samples, and data forms are labeled with a unique bar code identifier. A sample of the cord blood unit is evaluated for microbial contamination, sickle cell disease and HLA type. Characterization and safety testing are performed on the cord blood unit including nucleated cell count, viability, CD34⁺ cell count, potency, identity, and colony forming unit assays. hUCB units are cryopreserved using a solution of 10% DMSO and 1% dextran and stored in vapor phase of liquid nitrogen.

The hUCB compositions for use in the instant Example comprise hematopoietic progenitor cells, monocytes, lymphocytes, and granulocytes from human cord blood, which can be used for intravenous infusion. The potency of cord blood is determined by measuring the numbers of total nucleated cells (TNC) and CD34⁺ cells, and cell viability. Each unit of hUCB contains a minimum of 1×10⁷ total nucleated cells/kg in 25 mL at the time of cryopreservation. The cellular composition of hUCB depends on the composition of cells in the blood recovered from the umbilical cord and placenta of the donor. The actual nucleated cell count, the CD34⁺ cell count, the ABO group, and the HLA typing are listed in accompanying records sent with each individual unit.

The frozen hUCB units that have been collected and stored for bone marrow transplantation. The selected units are shipped to the study site. Before the shipping, the hUCB product release testing is performed. After being logged in at the study site, the hUCB is thawed at 37° C. and administrated in patients through IV infusion.

### II. Study Objectives and End Points

The primary objective of this Phase IIa study is to assess the safety and tolerability of hUCB infusion in patients with post-COVID. The secondary objective is to evaluate the efficacy of hUCB infusion in patients with post-COVID fatigue. In addition, the exploratory object is to assess the efficacy of hUCB infusion in improving quality of life and cognitive status in patients with post-COVID and to assess the change in biomarkers of cognitive impairment and fatigue.

The primary endpoint objective of this Phase IIa study is incidence of treatment-emergent adverse events (TEAEs). The secondary endpoint is to achieve changes of fatigue scores as measured by CFQ-11 from baseline (screening visit) to week 6, 12, 18 and 26 after treatment.

The exploratory endpoints include:
Change of fatigue score as measured by PGI-S from baseline (screening visit) to week 0, 3, 6, 8, 12, 18 and 26;
Change of FRAIL scale from baseline (screening visit) to week 0, 3, 6, 8, 12, 18 and 26;
Change in life quality as measured by EQ-5D-5L score from baseline (screening visit) to week 0, 3, 6, 8, 12, 18 and 26;
Change of cognitive score as measured by MoCA from baseline (screening visit) to week 12 and 26; and
Change in biomarkers of cognitive impairment and fatigue from baseline (week 0) to week 12 and 26.

### III. Study Design

### (a) General Design

This is a two-arm, multi-national, multi-center, single-blind, randomized, placebo-controlled study. A total of 30 patients with post-COVID are enrolled (active: placebo=2:1). Patients who have post-COVID are to be included in the study.

The study includes three periods: a screening period, a treatment period, and a follow-up period, with an overall study duration of approximately 30 weeks. Patients are to be administered one unit of either the hUCB composition or a placebo 3 times during the treatment period (at Week 0, 3 and 6) via intravenous infusion (IV). Patients are asked to stay at the study site for at least 2 hours post-dosing, or until the values return to baseline before discharge for safety observation. If patients develop serious allergic immune responses, they must be hospitalized for further observation.

The follow-up period includes 20 weeks (140 days, from week 7 to week 26). Remote monitoring through telephone or video calls is to be performed at week 8 and 18. The detailed time points of remote-monitoring and on-site visits are depicted in FIG. 1. Hard copies of fatigue-related, frailty and life quality questionnaires are provided to the patients, who are to be asked to answer these questionnaires at scheduled visits. On-site visits for the assessments of fatigue, frailty, life quality, cognitive deficits, and biomarkers of cognitive impairment and fatigue are required at week 12 and 26. See the Study Schedule provided in Table 1 below.

### (b) Scientific Rationale for Study Design

The hUCB composition used in the instant clinical study is a rich source of stem cells and progenitor cells that have beneficial effects on tissue repair due to CD133⁺ and CD34⁺ stem cells. Also, 20-40% of hUCB mononuclear cells are monocytes, which mediate immunosuppressive effects. Hence, hUCB is capable of allogeneic applying to resolve long-term illness such as cognitive impairment and fatigue in post-COVID syndrome or other illnesses such as neurodegenerative diseases.

The recommended minimum dose is at least 1×10⁷ total nucleated cells (TNC)/kg in this study, with a minimum total dose of 3×10⁷ TNC/kg per person in this study (three times of infusions are included).

### IV. Patient Criteria

### (a) Inclusion Criteria

An eligible patient must meet the following inclusion criteria:

$$Male or female aged \geq 18 and \leq 65 years . \qquad 1$$

2. With post-COVID syndrome, defined as signs and symptoms that develop during or after an infection consistent with COVID-19, continue for more than 6 months but not over 18 months, and are not explained by an alternative diagnosis. To ensure the above criteria are met, participants will be confirmed novel coronavirus (SARS-CoV-2) infection as determined by COVID-19 rapid antigen test or SARS-CoV-2 polymerase chain reaction (PCR) test at least 6 months prior to screening visit.
3. Has had a recent (within 7 days) negative SARS-CoV-2 test (an approved PCR or antigen test).
4. Meet the criteria for fatigue, based on the CFQ-11 case definition of fatigue.
5. Patient self-reported concerns regarding cognitive functioning or recent diagnosis of COVID-19 related cognitive impairment.

$$MoCA test scores \leq 28 / 30 at screening . \qquad 6.$$

7. Able to provide signed informed consent (by the patient or his/her legally authorized representative).
8. Is willing and able to participate in all aspects of the study, including completion of patientive evaluations, attendance at scheduled clinic visits, and compliance with all protocol requirements as evidenced by providing a written informed consent.
9. At least three ABO/Rh-matched study products are available for the patient.

### (b) Exclusion Criteria

Each patient also meets any of the following exclusion criteria at the time of treatment:
1. Persistent fatigue prior to COVID-19 diagnosis.
2. Neurological disorders prior to COVID-19 diagnosis.
3. Hospitalized in intensive care unit (ICU) within 1 year.
4. Known hypersensitivity to dimethyl sulfoxide (DMSO) or Dextran-40.
5. Had previously received stem cell therapy.
6. With pre-existing terminal illness.
7. With known immune disease.
8. Is pregnant or breastfeeding.
9. Has any concurrent or underlying disease or physical condition that, according to the investigator's evaluation, may affect the trial intervention or the patient's safety.
10. Is currently participating in another investigational study or has been taking any other investigational product within the last 4 weeks before screening.
11. Has received any vaccination within 3 weeks prior to the first hUCB infusion.
12. Under the conditions that may increase risk of complications based on the medical judgment of the investigator and the parameters as below:

$$Blood pressure systolic > 160 or < 90 mmHg or diastolic > 100 or < 60 mmHg$$

$$Pulse < 60 or > 105 bpm$$

$$Respiratory rate < 9 and > 28 bpm$$

$$Pulse oximetry < 94 \% in room air$$

$$Body temperature > 100.4 degrees Fahrenheit$$

Alanine aminotransferase (ALT) or aspartate aminotransferase (AST) >2 times the upper limit of normal (ULN)
Abnormal bilirubin unless patient has Gilbert's syndrome

$$Estimated glomerular filtration rate ( eGFR ) < 60 mL / min / 1.73 {m}^{2} by Chronic Kidney Disease Epidemiology Collaboration ( CKD - EPI )$$

$$Hemoglobin < 10 mg / dL$$

$$Platelet count < 100 , 000 / μ L$$

13. Presence of medical condition(s) during the as below:
   Chronic heart failure (CHF) in New York Heart Association (NYHA) class II to IV
   Chronic kidney disease (CKD) in stage 3b to 5
   Acute respiratory distress syndrome (ARDS)
   Psychiatric condition that is likely to interfere with the conduct of the study (e.g., chronic depression)
   Chronic pulmonary disease
14. Having been diagnosed abuse of alcohol or drugs

### V. Study Treatment

Each unit of hUCB composition contains a minimum of 1.0×10⁷ TNC/kg suspended in 10% DMSO and 1% Dextran 40, at the time of cryopreservation. Matching for ABO/Rh are required. No irradiation is performed to the hUCB composition.

(a) hUCB treatment

The hUCB is prepared for infusion following conventional operating procedures. Briefly, the hUCB is thawed at 37° C. and administrated to the patient by IV infusion within 20 minutes after thawing. The limit on DMSO administration may be 1 g/kg body weight per day. The patient is not administered via the same tubing products other than 0.9% Sodium Chloride, Injection (USP). When needed, the hUCB composition may be filtered through a 170 to 260 micron filter designed to remove clots. Filters designed to remove leukocytes should not be used.

It is recommended that the infusion rate be 1.5-2 mL per minute for adults. One cyrobag of hUCB infusion can be administrated in about 20 minutes. The infusion rate should be reduced if the fluid load is not tolerated. The infusion should be discontinued in the event of an allergic reaction or if the patient develops a moderate to severe infusion reaction.

### (b) Management of Adverse Reactions

Occurrence of adverse reactions is monitored during administration of the hUCB composition and for at least two hours after administration, or until the values return to baseline before discharge.

Acute allergic reactions are inherent, though rare, consequences of dosing with biological agents. If any adverse reactions occurs during infusion, the infusion rate may be reduced or the infusion may be discontinued. hUCB infusion may resume after the adverse reaction(s) is well-managed. If a patient develops serious allergic immune responses, that patient shall be hospitalized for further observation.

A patient should discontinue the treatment when any of the following occurs:
1. Worsening of the post-COVID conditions (fatigue, cognitive impairment, or life quality) due to COVID-19 reinfection
2. Severe infusion or allergic reactions occur that requiring medical intervention
3. A suspected hUCB-related AE of CTCAE version 5.0 grade 3 or higher
4. Any hUCB-related SAE
5. Pregnancy

### (c) Concomitant Medications/Treatments

### Permitted Medications/Treatments

If the administration of any concomitant treatment is deemed necessary minimum changes (dose, frequency, etc.) should be made to the concomitant treatment during the study. However, concomitant medication use should be kept to a minimum during the study.

Vaccination is prohibited within the duration from three weeks prior to the first hUCB infusion to three weeks following the last hUCB infusion. No further restriction on vaccination is imposed thereafter.

### Prohibited Medications/Treatments

Any other cell therapy is prohibited during the study.

Vaccination is prohibited within the duration from three weeks prior to the first hUCB infusion to three weeks following the last hUCB infusion.

### VI. Study Procedures

The study schedule provided in Table 1 below summarizes the trial procedures to be performed at each visit. There are 6 on-site visits and 2 remote monitoring.

TABLE 1
Study Schedule

| | Phase | | | | | | | |
|---|---|---|---|---|---|---|---|---|
| | Screening | Treatment | | | Follow-up | | | |
| | Visit | | | | | | | |
| | | | 3 | 4/EOT | | | | |
| | | | 22 ± 3 | 43 ± 3 | | | | |
| | 1 | 2 | (W 3) | (W 6) | 5 a | 6 | 7 a | 8/EOS |
| | −28 to −1 | 1 | V2 + | V3 + | 57 ± 3 | 85 ± 7 | 127 ± 7 | 183 ± 7 |
| | (W −4 to −1) | (W 0) | 21 days | 21 days | (W 8) | (W 12) | (W 18) | (W 26) |
| Informed consent | X | | | | | | | |
| Inclusion/exclusion | X | X | | | | | | |
| criteria | | | | | | | | |
| Medical history, | X | | | | | | | |
| diagnosis, treatment | | | | | | | | |
| history, and | | | | | | | | |
| demographics ^{b} | | | | | | | | |
| Serum pregnancy test | X | | | | | | | |
| (In case of a female | | | | | | | | |
| patient) ^{c} | | | | | | | | |
| Urine pregnancy test | | X | X | X | | X | | X |
| (In case of a female | | | | | | | | |
| patient) ^{c} | | | | | | | | |
| Physical | X | X | X | X | | X | | X |
| examination/vital | | | | | | | | |
| signs ^{d} | | | | | | | | |
| Pulse oximetry | X | | | | | | | |
| Laboratory- | X | X | X | X | | X | | X |
| Hematology ^{e} | | | | | | | | |
| Laboratory- | X | X | X | X | | X | | X |
| Biochemistry ^{f} | | | | | | | | |
| Laboratory- | | X | | | | X | | X |
| Biomarkers ^{g} | | | | | | | | |
| Laboratory- | X | X | X | X | | X | | X |
| Urinalysis ^{h} | | | | | | | | |
| HLA typing ⁱ | X | | | | | | | |
| Randomization | X | | | | | | | |
| ABO/Rh-matching | X | | | | | | | |
| IP administration | | X | X | X | | | | |
| CFQ-11 ^{j} | X | | | X | | X | X | X |
| PGI-S, FRAIL, | X | X | X | X | X | X | X | X |
| EQ-5D-5L ^{k} | | | | | | | | |
| MoCA ^{l} | X | | | | | X | | X |
| Exit interview ^{m} | | | | | | | | X |
| Adverse events | | X | X | X | X | X | X | X |
| Concomitant | | X | X | X | X | X | X | X |
| medication/therapy ^{b} | | | | | | | | |

a. Remote monitoring through telephone or video calls will be performed at week 8 and 18. Patients will be provided with hard copies of fatigue-related, frailty, and life quality questionnaires at EOT (W 6) and week 12. The study staff shall contact the patients to remind them to complete all necessary assessments on the day before the scheduled visit and/or on the day of the scheduled visit. During remote monitoring, the study staff shall inquire about the patient' status and record AE and concomitant medication information, if any.
^{b} Medical or medication/treatment history within 1 year prior to W 0 should be recorded. Any medication or therapy during study period will be regarded as concomitant medication/therapy and should be documented.
^{c} Serum or urine pregnancy test is only for female patients of childbearing potential. Urine or serum pregnancy test should be done in patients of childbearing potential and should be negative prior to administration of study product.
^{d} Blood pressure, pulse rate, respiration rate, and body temperature. On the days of IP infusion, vital signs and physical examination should be done within 30 minutes prior to IP infusion, end of IP infusion, and then every 1 hour till 2 hours post-infusion, or until the values return to baseline before discharge for safety observation. Data collected at W 0 will serve as baseline.
^{e} Hemoglobin (Hb), hematocrit (Hct), red blood cell (RBC) count, white blood cell (WBC) count and WBC differential, platelet, prothrombin time (PT), and activated partial thromboplastin time (aPTT). Samples for hematology tests will be collected prior to IP infusion at week 0, 3, and 6. Data collected at W 0 will serve as baseline.
^{f} Alanine transaminase (ALT), aspartate transaminase (AST), alkaline phosphatase (ALP), albumin, total bilirubin, sodium (Na), potassium (K), glucose, creatinine, blood urea nitrogen (BUN) and estimated glomerular filtration rate (eGFR) calculated by Chronic Kidney Disease Epidemiology Collaboration (CKD-EPI). Samples for biochemistry tests will be collected prior to IP infusion at week 0, 3 and 6. Data collected at W 0 will serve as baseline.
^{g} Efficacy-related biomarkers at W 0, W 12 and W 26 should be recorded, such as brain-derived neurotrophic factor [BDNF] and/or other future biomarker related to cognitive impairment and fatigue. At W 0, samples will be collected before IP infusion.
^{h} Color, appearance, specific gravity, pH, protein, glucose, occult blood, ketone, leukocyte esterase, nitrite, bilirubin, urobilinogen, urine sediment via microscopic assessment. Samples for urinalysis will be collected prior to IP infusion at week 0, 3, and 6. Data collected at W 0 will serve as baseline.
ⁱ HLA-antigen typing: Low-resolution typing will be performed for HLA-A and HLA-B; high-resolution typing will be performed for DRB1.
^{j} Fatigue-related questionnaires: Chalder fatigue scale (CFQ-11). CFQ-11 will be collected at screening visit, week 6, 12, 18, and 26 (each time

In Table 1, CFQ-11 refers to Chalder fatigue scale; EOS refers to end of study; EOT refers to end of treatment; EQ-5D-5L refers to EuroQoL 5-Dimension 5-Level; IP refers to investigational product (human umbilical cord blood composition); MoCA refers to Montreal cognitive assessment; PGI-S refers to Patient Global Impression of Severity; and W refers to week.

### (a) Screening Visit (Visit 1; Day −28 to −1)

The following assessments are to be performed to determine eligibility:
Obtain demographic information.
Review medical, diagnosis and medication/treatment history to determine eligibility based on inclusion/exclusion criteria and document comorbidities and related information.
Confirm eligibility criteria as provided herein.
Document post-COVID conditions.
Perform physical examination.
Assess vital signs.
Perform pulse oximetry test.
Collect blood samples for hematology and biochemistry tests.
Collect urine samples for laboratory tests.
Perform serum pregnancy test (only for female patients of childbearing potential).
Collect specimen for ABO/Rh and HLA typing.
Perform randomization.
Complete CFQ-11, PGI-S, FRAIL scale, EQ-5D-5L, and MoCA questionnaires.

### (b) Treatment Visit (Visit 2; Day 1/Week 0)

The following procedure will be performed on Day 1:
Pre-infusion:
   Confirm eligibility criteria
   Perform physical examination
   Assess vital signs (within 30 minutes prior to infusion)
   Perform urine pregnancy test (only for female patients of childbearing potential)
   Collect blood samples for hematology and biochemistry tests, as well as laboratory tests for biomarkers
   Collect urine samples for laboratory tests
   Prepare for IP infusion
   Complete PGI-S, FRAIL scale, EQ-5D-5L questionnaires
   Record concomitant medication/therapy
Post-infusion:
   Perform physical examination and assess vital signs (at the end of infusion and then every 1 hour till 2 hours post-infusion hours, or until the values return to baseline before discharge)
   Monitor and record AE
   Record concomitant medication/therapy
   Dispense Discharge Instructions

### (c) During Treatment Period (Visit 3 and 4; Week 3 and 6)

The following procedure will be performed on Day 22 and Day 43:
Pre-infusion
   Perform physical examination
   Assess vital signs (within 30 minutes prior to infusion)
   Perform urine pregnancy test (only for female patients of childbearing potential)
   Collect blood samples for hematology and biochemistry tests
   Collect urine samples for laboratory tests
   Prepare for hUCB infusion
   Complete CFQ-11 questionnaire (only at Week 6)
   Complete PGI-S, FRAIL scale, EQ-5D-5L questionnaires
   Record concomitant medication/therapy
Post-infusion:
   Perform physical examination and assess vital signs (at the end of infusion and then every 1 hour till 2 hours post-infusion, or until the values return to baseline before discharge)
   Monitor and record AE
   Record concomitant medication/therapy
   Dispense Discharge Instructions

Patients will be provided with hard copies of fatigue-related, frailty, and life quality questionnaires (PGI-S, FRAIL scale, EQ-5D-5L) at Week 6 (end of treatment, EOT) for the remote visit at Week 8.

### (d) Follow-Up Visits (Visit 5 Through 7)

Day 85 (Week 12): On-site visits are required on Day 85.

The following procedure will be performed:
Perform physical examination
Assess vital signs
Perform urine pregnancy test (only for female patients of childbearing potential)
Collect blood samples for hematology and biochemistry tests, as well as laboratory tests for biomarkers
Collect urine samples for laboratory tests
Collect PGI-S, FRAIL scale, EQ-5D-5L questionnaires completed at Week 8
Complete CFQ-11, PGI-S, FRAIL scale, EQ-5D-5L questionnaires
Instructed to complete MoCA questionnaire
Record AE
Record concomitant medication/therapy

Patients will be provided with hard copies of fatigue-related, frailty, and life quality questionnaires (CFQ-11, PGI-S, FRAIL scale, EQ-5D-5L) at Week 12 for the remote visit at Week 18.

### Day 57 and Day 127 (Week 8 and 18, Remote Monitoring)

Remote monitoring through a telephone or video call is performed. The following procedure is performed:
Complete CFQ-11 questionnaire (only at Week 18)
Complete PGI-S, FRAIL scale, EQ-5D-5L questionnaires
Record AE
Record concomitant medication/therapy

### (e) End of Study (Visit 8; Day 183/Week 26)

Patients are required to return to the study site for an EOS visit. The following procedure is performed:
Perform physical examination
Assess vital signs
Perform urine pregnancy test (only for female patients of childbearing potential)
Collect blood samples for hematology and biochemistry tests, as well as laboratory tests for biomarkers
Collect urine samples for laboratory tests
Collect CFQ-11, PGI-S, FRAIL scale, EQ-5D-5L questionnaires completed at Week 18
Complete CFQ-11, PGI-S, FRAIL scale, EQ-5D-5L questionnaires
Instructed to complete MoCA questionnaires
Conduct exit interview
Record AE
Record concomitant medication/therapy

### VII. Study Evaluations

### (a) Demography Data and Medical History

The following demography data and medical history are to be evaluated.
1. Date of Birth
2. Sex: Male or Female
3. Ethnicity: Hispanic/Latino or Not Hispanic/Latino
4. Race: American Indian/Alaska Native, Asian, Black/African American, Native Hawaiian/Other Pacific Islander, or White
5. Height, Weight, and Body Mass Index (BMI): Height is measured for the patient not wearing shoes at screening. The measurement of height will be rounded to the nearest tenth of a centimeter. Body weight is measured for the patient not wearing shoes but wearing light clothing at screening. The measurement of weight will also be rounded to the nearest tenth of a kilogram. (If this procedure, measuring the height and weight, cannot be performed, obtaining this information from patients' medical charts [within 1 month] is acceptable).
6. Significant Medical History: COVID-19-related (every event) or non-COVID-19-related (within 1 year prior to week 0).
   Date of COVID-19 diagnosis/confirmation by PCR test or antigen test
   Date of symptom onset
7. Medication History: Any medication (taken within 3 months, including over-the-counter or prescription medicines, vitamins, or herbal supplements), therapy, or vaccine (received within 1 year) prior to week 0, categorized into COVID-19-related or non-COVID-19-related.
8. ABO/Rh-Matching: Only patients with ABO/Rh-matched hUCB are allowed to enter the study
9. HLA typing: Low-resolution typing will be performed for HLA-A and HLA-B; high-resolution typing will be performed for DRB1. Low-resolution typing is by antibodies or RT/PCR of mRNA of known HLA sequences. High-resolution typing is by sequencing of HLA genes.
10. Post-COVID Status
   Date of last sample collection for COVID-19 PCR test or antigen test as well as the test result
   Duration of symptoms
   Symptoms at entry: such as fever, cough, headache, fatigue, anorexia, problems with memory and concentration (brain fog), difficulty sleeping (insomnia), dizziness, depression and anxiety, shortness of breath, sore throat, nasal congestion, gastrointestinal symptoms, loss of smell (anosmia), loss of taste (ageusia), or other symptoms associated with post-COVID, if applicable
11. Fatigue “case” or “non-case”: the condition of fatigue will be based on CFQ-11 case definition of fatigue.
12. Cognitive Function: cognitive score measured by MoCA test.

### (b) Vital Signs

1. Vital sign measurements include blood pressures (systolic blood pressure [SBP] and diastolic blood pressure [DBP]), pulse rate, respiratory rate, and body temperature. Vital signs will be assessed at each visit as outlined in Table 1. Fever is defined as body temperature greater than 38° C. (100.4° F.) (surface temperature greater than 37.2° C. (98.96° F.) measured by non-contact infrared thermometers [NCITs]).
2. Measurements should be taken in a seated position after resting for approximately 10 minutes. The same thermometer should be used consistently throughout the study.
3. On the days of hUCB infusion, vital signs should be done within 30 minutes prior to hUCB infusion, end of IP infusion, and then every 1 hour till 2 hours post-infusion, or until the values return to baseline before discharge for safety observation.

### (c) Pulse Oximetry Test

Oxygen level (oxygen saturation) of the blood should be measured via pulse oximeter. Pulse oximetry test is performed at screening to confirm patient's eligibility into this study.

### (d) Physical Examinations

1. A complete physical examination is conducted at screening. Complete physical examination items include general appearance, HEENT (head, eyes, ears, nose, and throat), mouth, skin, neck (including thyroid), lymph nodes, spine, cardiovascular system, respiratory system, gastro-intestinal system, nervous system, musculoskeletal system, blood and blood-forming organs, mental status, and other body systems if applicable for describing the status of patient's health. At subsequent visits, an abbreviated physical examination comprising a brief medical history and targeted physical examination is performed to evaluate possible adverse events.
2. On the days of hUCB infusion, physical examination should be done within 30 minutes prior to hUCB infusion, end of hUCB infusion, and then every 1 hour till 2 hours post-infusion, or until the values return to baseline before discharge for safety observation.
3. Any significant physical examination findings after dosing is regarded as adverse events.

### (e) Laboratory Evaluations

Laboratory tests (including hematology, biochemistry, urinalysis, and biomarkers) will be performed at scheduled visits as outlined in Table 1. The examination items include analytes in Table 2 below:

TABLE 2
Analytes for Examination

| | |
|---|---|
| Hematology | hemoglobin (Hb), hematocrit (Hct), red blood cell (RBC) |
| | count, white blood cell (WBC) count and WBC differential, |
| | platelet, prothrombin time (PT), and activated partial |
| | thromboplastin time (aPTT) |
| Biochemistry | alanine transaminase (ALT), aspartate transaminase (AST), |
| | alkaline phosphatase (ALP), albumin, total bilirubin, |
| | sodium (Na), potassium (K), glucose, creatinine, blood |
| | urea nitrogen (BUN) and estimated glomerular filtration |
| | rate (eGFR) |
| Urinalysis | color, appearance, specific gravity, pH, protein, |
| | glucose, occult blood, ketone, leukocyte esterase, |
| | nitrite, bilirubin, urobilinogen, urine sediment via |
| | microscopic assessment. |
| Biomarkers | brain-derived neurotrophic factor [BDNF] and/or other |
| | future biomarker related to cognitive impairment and |
| | fatigue |

Additional tests may be conducted at any time during the study as determined necessary. A clinically significant change from baseline is recorded as an AE.

eGFR is calculated using the CKD-EPI equation:

$$eGFR = 141 \times {min ( Scr / κ , 1 )}^{α} \times max ( Scr / κ , 1 ) - 1.209 \times {0.993}^{A} {g}^{e} \times 1.018 [ if female ] or \times 1.159 [ if African American ]$$

Scr is serum creatinine in μmol/L,
κ is 61.9 for females and 79.6 for males,
α is −0.329 for females and −0.411 for males,
min indicates the minimum of Scr/κ or 1, and
max indicates the maximum of Scr/κ or 1

### (f) Fatigue Assessment

### Cfq-11 Questionnaire

Fatigue is assessed using CFQ-11 questionnaire. Patients are instructed to complete the CFQ-11 questionnaire as delineated in Table 1. CFQ-11 is a self-administered questionnaire for measuring the extent and severity of fatigue. It generally spans two dimensions: physical fatigue (questions 1-7) and mental fatigue (questions 8-11). Good internal consistency has been confirmed by a split half reliability of 0.85 and a Cronbach alpha that ranges between 0.86 and 0.92. Chalder, J. Psychosomatic Research 2010; 69(1): 17-22; Chalder et al., Journal of Psychosomatic Research 1993; 37(2): 147-53; and Loge et al., Journal of Psychosomatic Research 1998; 45(1): 53-65. Reliability and validation of CFQ-11 has also been demonstrated. De Vries et al., Occupational and Environmental Medicine 2003; 60(suppl 1): i10-i5.

CFQ-11 has two scoring system, the bimodal scoring system (the respondents answer the question with 1 or 0) and the Likert scoring system (responses on the extreme left receive a score of 0, increasing to 1, 2 or 3 as they become more symptomatic). The scoring scheme of CFQ-11 is listed in Table 3 below.

TABLE 3
Scoring Scheme of CFQ-11

| | Bimodal score | Likert Score |
|---|---|---|
| Less than usual | 0 | 0 |
| No more than usual | 0 | 1 |
| More than usual | 1 | 2 |
| Much more than usual | 1 | 3 |

CFQ-11 allows the differentiation of “cases” vs “non-cases” using the bimodal scoring method with a fatigue score ranging from 0 to 11. Patients with a score 4 or more are defined as cases of fatigue. Total CFQ-11 score (from a maximum of 33) using Likert scoring method is used for the assessment of fatigue severity. See, e.g., goodmedicine.org.uk/files/assessment,%20chalder %20fatigue %20scale.pdf, the relevant disclosure of which is incorporated by reference for the subject matter and purpose referenced herein.

### Pgi-S Questionnaire

The severity of fatigue is assessed using PGI-S questionnaire. Patients are instructed to complete the PGI-S questionnaire as delineated in Table 1.

PGI-S for fatigue is a 1-item questionnaire for measuring the severity of fatigue (see, e.g., fda.gov/media/116281/download, the relevant disclosures of which are incorporated by reference for the subject matter and purpose referenced herein). PGI-S is a reliable measurement tool for fatigue severity validated with cognitive and physical functioning questionnaire.

### (g) FRAIL Scale

Frailty is assessed by FRAIL questionnaire. Patients are instructed to complete the FRAIL questionnaire as delineated in Table 1 above.

The FRAIL scale was first proposed by International Association of Nutrition and Ageing (IANA), and it includes five components: Fatigue, Resistance, Ambulation, Illness, and Loss of weight. van Kan et al., The Journal of Nutrition Health and Aging 2008; 12(1): 29-37; and van Kan et al., Journal of the American Medical Directors Association 2008; 2(9): 71-2. FRAIL scores range from 0 to 5 (i.e., 1 point for each component; 0=best to 5=worst) and represent frail (3-5), pre-frail (1-2), and robust (0) health status.

The 5 components and how they are being assessed are listed in Table 4 below.

TABLE 4
Components of FRAIL Scale

| Components | Assessments |
|---|---|
| Fatigue (F) | asking respondents how much time during the past 4 |
| | weeks they felt tired with responses of “all of the |
| | time” or “most of the time” scored 1 point. |
| Resistance | asking participants if they had any difficulty walking |
| (R) | up 10 steps alone without resting and without aids; |
| | “yes” response was scored as 1 point. |
| Ambulation | asking if they had any difficulty walking several |
| (A) | hundred yards alone and without aids; “yes” response |
| | was scored as 1 point. |
| Illness (I) | scored 1 for respondents who reported 5 or more |
| | illnesses out of 11 total illnesses. |
| Loss of | scored 1 for respondents with a weight decline of 5% or |
| weight (L) | greater within the past 12 months based on self-report. |

The FRAIL scale has been demonstrated as a valid predictor for mortality based on a community-based cohort and is a potentially useful frailty screening tool. Thompson et al., Australasian Journal on Ageing 2020; 39(4): e529-e36.

### (h) Life Quality Assessment

Patients are instructed to complete the EQ-5D-5L questionnaire as delineated in Table 1 above.

The EQ-5D-5L is a validated questionnaire to evaluate patients' quality of life by assessment of the following five factors: mobility, self-care, usual activities, pain or discomfort, and anxiety or depression. Categorization within each factor is divided into five levels that range from no problems to extreme problems. Herdman et al., Quality of Life Research 2011; 20(10): 1727-36. The details of EQ-5D-5L questionnaire can be found at, e.g., euroqol.org/information-and-support/euroqol-instruments/eq-5d-51/, the relevant disclosures of which are incorporated by reference for the subject matter and purpose referenced herein.

### (i) Cognitive Deficits-Related Assessment

Patients are instructed to complete the MoCA questionnaire as outlined in Table 1 above.

The MoCA is a one-page 30-point test administered in approximately 10 minutes. It is a widely used cognitive screening tool with high sensitivity and specificity. Nasreddine et al., Journal of the American Geriatrics Society 2005; 53(4): 695-9. The MoCA total score is comprised of 30 points for items categorized into seven domains:
(1) visuospatial/executive;
(2) naming;
(3) attention;
(4) language;
(5) abstraction,
(6) memory and
(7) orientation.

The details of MoCA questionnaire and how the scores are calculated following convention methodology. Any of versions v8.1, v8.2, and v8.3 can be used. People who score 26 or higher are generally considered to be functioning normally.

To avoid the learning effect caused by repeat assessment in a short period of time, three different versions of MoCA questionnaire are available for the study. Each version must be used once only for each patient.

### (j) Adverse Events

The patient are asked to report AE voluntarily. The methods and procedures for recording, assessing, and reporting AEs are described above.

### (k) Concomitant Medications and Therapies

All medications and therapies administered or taken by the patient starting from week 0 throughout the study are considered as concomitant medications/therapies. Patients must be questioned at each visit concerning any new medications or changes in current medications including over-the-counter medication and topical medication.

### (l) Exit Interview

An exit interview is incorporated at EOS visit to improve the development program of hUCB infusion treatment for post-COVID. All patients are asked about their willingness and invited to participate in a semi-structured interview via ICF. Patients who consent to participate are asked by a trained interviewer about their overall improvement in health and quality of life (in work, driving, study, housework, family, and leisure activities), and drug infusion experience. The sponsor will provide an instruction manual and training for the interviewer before study initiation. The questions to be asked are as listed in Table 5 below.

TABLE 5
Questions for Exit Interview

1. How would you rate the overall improvement in health or quality of
life after receiving the study treatment in the following activities?
(Please choose one item for each activity)

| | |
|---|---|
| Work/Study | L111. Very much better |
| | L112. Much better |
| | L113. Moderately better |
| | L114. A little better |
| | L115. Almost the same as before |
| Driving/Commuting | L111. Very much better |
| | L112. Much better |
| | L113. Moderately better |
| | L114. A little better |
| | L115. Almost the same as before |
| Housework | L111. Very much better |
| | L112. Much better |
| | L113. Moderately better |
| | L114. A little better |
| | L115. Almost the same as before |
| Family | L111. Very much better |
| | L112. Much better |
| | [1113. Moderately better |
| | [1114. A little better |
| | [1115. Almost the same as before |
| Leisure activities | [1111. Very much better |
| | [1112. Much better |
| | [1113. Moderately better |
| | [1114. A little better |
| | [1115. Almost the same as before |

2. What would you like to improve the most in post-COVID condition?
3. Have you suffered from any of the following discomforts
before the drug infusion?
[1111. Sleep disorder/Unrefreshing sleep
[1112. Inability to remain upright posture and prefer to lie down
[1113. Inability to exercise/reduced activity due to fatigue
[1114. Poor memory/attention
[1115. None of above
4. Any of the discomforts in Question 3 that you think have
alleviated or recovered after the drug infusion?
[1111. Sleep disorder/Unrefreshing sleep
[1112. Inability to remain upright posture and prefer to lie down
[1113. Inability to exercise/reduced activity due to fatigue
[1114. Poor memory/attention
[1115. None of above
5. How would you rate the drug infusion experience?
□1. Highly satisfied
□2. Satisfied
□3. Average
□4. Dissatisfied
□5. Highly dissatisfied
6. What would you suggest for the drug infusion experience?

### VIII. Assessment of Safety

### (a) Adverse Events (AE)

An AE is any untoward or unfavorable medical occurrence in a human patient, including any abnormal sign (for example, abnormal physical exam or laboratory finding), symptom, or disease, temporally associated with the patient's participation in the research, whether or not considered related to the patient's participation in the research.

Information to be collected includes event description, time of onset, investigator's assessment of severity, relationship to study product (assessed only by those with the training and authority to make a diagnosis) and time of resolution/stabilization of the event. All AEs occurring while on study must be documented appropriately regardless of relationship. All AEs will be followed to adequate resolution.

Pre-existing conditions are recorded as baseline on the “Medical History.” If the pre-existing condition does not change, it does not have to be reported as an AE on subsequent cycles. However, if it deteriorates at any time during the study, it is recorded as an AE. Any abnormal laboratory test results (hematology, biochemistry, and urinalysis) or other safety assessments (e.g., physical examination, and vital sign measurements), including those that worsen from baseline, that are considered to be clinically significance in the medical and scientific judgement of the investigator need to be recorded as AEs or SAEs. Abnormal laboratory assessments that are considered a symptom of an underlying AE or part of a syndrome that is reported as AE (e.g., presence of blood cells in urine in a person diagnosed with urinary tract infection) do not need to be documented additionally as AE, but a comment should be added to the underlying AE and the abnormal laboratory value.

All AEs must be graded for severity and relationship to study product.

### Severity of AEs

All AEs will be assessed by the clinician using the Common Terminology Criteria for Adverse Events (CTCAE) version 5.0. For events not included in the protocol defined grading system, the following guidelines in Table 6 below are to be used to quantify intensity.

TABLE 6
Guidelines for Adverse Events

| | |
|---|---|
| Grade 1 | Mild; asymptomatic or mild symptoms; clinical or diagnostic |
| | observations only; |
| | intervention not indicated. |
| Grade 2 | Moderate; minimal, local, or noninvasive intervention |
| | indicated; limiting age-appropriate instrumental activities |
| | of daily living (ADL). |
| | (Instrumental ADL refer to preparing meals, shopping for |
| | groceries or clothes, using the telephone, managing money, etc.) |
| Grade 3 | Severe or medically significant but not immediately |
| | life-threatening; hospitalization or prolongation of |
| | hospitalization indicated; disabling; limiting self-care ADL. |
| | (Self-care ADL refer to bathing, dressing, and undressing, |
| | feeding self, using the toilet, taking medications, and |
| | not bedridden.) |
| Grade 4 | Life-threatening consequences; urgent intervention indicated. |
| Grade 5 | Death related to AE. |

### Relationship to Study Products

All AEs must have their relationship to study product assessed using the terms defined as follows. In a clinical trial, the study product must always be suspect.

The relationship between the use of IP and the occurrence of each AE must be assessed and categorized by the investigator using clinical judgment to determine the causal relationship. Other factors such as medical history of underlying diseases, concomitant medication/therapy and any other relevant risk factors should be considered.

To help assess, the following guidelines are used:

### Causality

Definitely/Certain: There is clear evidence to suggest a causal relationship, and other possible contributing factors can be ruled out. The clinical event, including an abnormal laboratory test result, occurs within a reasonable timeframe after study procedure(s) and cannot be explained by concurrent disease or other drugs or chemicals.
Probable/Likely: There is evidence to suggest a causal relationship, and the influence of other factors is unlikely. The clinical event, including an abnormal laboratory test result, occurs within a reasonable time after study procedure(s), is unlikely to be attributed to concurrent disease or other drugs or chemicals, and follows a clinically reasonable response to study events.
Possible: There is some evidence to suggest a causal relationship (e.g., the event occurred within a reasonable time after study procedure(s)). However, the influence of other factors may have contributed to the event (e.g., the patient's clinical condition, other concomitant events). Although an adverse event may be judged only as “possibly related” soon after discovery, it can be flagged as requiring more information and later be upgraded to “probably related” or “definitely related”, as appropriate.
Unlikely: A clinical event, including an abnormal laboratory test result, whose temporal relationship to study procedure(s) makes a causal relationship improbable (e.g., the event did not occur within a reasonable time after study procedure(s)) and in which other drugs or chemicals or underlying disease provides plausible explanations (e.g., the participant's clinical condition, other concomitant treatments).
Unrelated: The AE is completely independent of study procedure(s), and/or evidence exists that the event is definitely related to another cause. There must be an alternative, definitive cause documented by the clinician.

In some particular cases, the event may not be adequately assessed because information is insufficient or contradictory and/or the data cannot be verified or supplemented. The investigator will make the maximum effort to define and categorize the event and avoid these situations. Where the investigator remains uncertain about classifying the event, it should not exclude the relatedness and should classify the event as “possible”.

### (b) Expected Adverse Reactions

Diagnosis of acute adverse transfusion reactions is based on the recognition of the signs and symptoms by the bedside. Common presentation and differential diagnosis are listed in Table 7 below. See also Suddock et al., Transfusion reactions. StatPearls [Internet]: StatPearls Publishing; 2021.

TABLE 7
Clinical Presentation and Diagnosis

| Clinical | |
|---|---|
| presentation | Differential diagnosis |
| Fever/Chills | Fever and/or chills are most commonly associated |
| | with a febrile, non-hemolytic reaction, however; |
| | they can also be the first sign of a more serious |
| | acute hemolytic reaction, TRALI, or septic |
| | transfusion reaction. |
| | If the temperature rises 1° C. or higher from the |
| | temperature at the start of transfusion, the |
| | transfusion should be stopped. Acute hemolytic |
| | reaction or bacterial contamination should be |
| | suspected if there is a greater rise in temperature, |
| | or more serious symptoms (e.g., rigors). |
| Urticaria/Itching | Urticaria (hives) and/or itching can be the |
| | presenting sign of a mild allergic reaction, but |
| | can also be associated with the onset of a life- |
| | threatening anaphylactic reaction. The transfusion |
| | should be stopped, and the patient should be |
| | carefully monitored for progression of symptoms. |
| Respiratory | Dyspnea, or shortness of breath, is a concerning |
| Distress/Dyspnea | sign that can often be seen with more severe |
| | reactions including anaphylaxis, TRALI, and |
| | TACO. It can also be seen by itself without |
| | accompanying symptoms. |
| Hypotension | Hypotension can be seen with an acute hemolytic |
| | reaction, septic transfusion reactions, anaphylaxis, |
| | and TRALI. They have also been reported without |
| | the presence of any other associated transfusion |
| | reaction. |
| Hypothermia | Hypothermia can be seen with large volume |
| | transfusions of refrigerated products. The only |
| | intervention needed is warming the patient and/or |
| | blood product. |

TRALI, transfusion related acute lung injury; TACO, transfusion-associated circulatory overload.

In reference to similar products of HPC, Cord Blood, approved by the US FDA, infusion reactions are expected to occur and include nausea, vomiting, fever, rigors or chills, flushing, dyspnea, hypoxemia, chest tightness, hypertension, tachycardia, bradycardia, dysgeusia, hematuria, and mild headache. Premedication with antipyretic, histamine antagonists, and corticosteroids may reduce the incidence and intensity of infusion reactions. Severe infusion reactions, including respiratory distress, severe bronchospasm, severe bradycardia with heart block or other arrhythmias, cardiac arrest, hypotension, hemolysis, elevated liver enzymes, renal compromise, encephalopathy, loss of consciousness, and seizure also may occur.

Allergic reactions may also occur after hUCB infusion, including bronchospasm, wheezing, angioedema, pruritus, and hive. Given that clinical trials are conducted under widely varying conditions, adverse reaction rates observed in the clinical trials of a drug cannot be directly compared to rates in the clinical trials of another drug and may not reflect the rates observed in practice.

The recommended premedication regimen before infusion is provided as follows:
1. Antipyretic:
Acetaminophen (TYLENOL, tablets, 650 mg, oral, once, for one dose), administer 30 to 60 minutes prior to transfusion.
2. Histamine antagonists:
   Diphenhydramine (BENADRYL, tablets, 25 mg, oral or intravenous injection, once, for one dose), administer 30 to 60 minutes prior to transfusion.
   Famotidine (PEPCID, tablets, 20 mg, oral or intravenous injection, once, for one dose), administer 30 to 60 minutes prior to transfusion.
   Fexofenadine (ALLEGRA, tablets, 180 mg, oral, once, for one dose), administer 30 to 60 minutes prior to transfusion.
3. Corticosteroids:
   Hydrocortisone sodium succinate (Solu-CORTEF, 100 mg, intravenous injection, once, for one dose), administer 30 to 60 minutes prior to transfusion.
   Methylprednisolone sodium succinate (Solu-MEDROL, 10 mg, intravenous injection, once, for one dose), administer 30 to 60 minutes prior to transfusion.

Patients are given premedication prior to transfusion. Precautions of these pre-transfusion medications are:
1. Take premedication either alone or in combination.
2. Use one of a kind for each category of medication per patient.
3. Patients with known allergies to any drug mentioned above are not included in this premedication regimen.

### (c) Serious Adverse Events (SAE)

An SAE is any AE/experience occurring that results in any of the following outcomes:
Death
Life-threatening (defined as an event in which the patient is at immediate risk of death at the time of the event; life-threatening does not refer to an event that hypothetically might have caused death if it were more severe, resulting in permanent or significant disability/incapacity)
Requires inpatient hospitalization or prolongation of existing inpatient hospitalization
Results in congenital anomaly or birth defect
Results in persistent or significant disability or incapacity
Events requiring medical and/or surgical intervention to prevent permanent impairment of function or permanent damage to a body structure

Events that are expected to be due to the progression of underlying post-COVID condition will be collected as AEs but will not be reported as SAE, unless the event is serious and the investigator deems it to be related to the administration of the hUCB.

Hospitalization or prolongation of hospitalization in the absence of a precipitating, clinical AE is not in itself an SAE. Examples include:
Admission for treatment of a preexisting condition not associated with the development of a new AE or with a worsening of the preexisting condition (e.g., for work-up of persistent pretreatment lab abnormality)
Administrative admission (e.g., for yearly physical exam)
Protocol-specified admission during a clinical trial (e.g., for a procedure required by the trial protocol)
Preplanned treatments or surgical procedures should be noted in the baseline documentation for the entire protocol and/or for the individual patient
Hospitalization for the purpose of isolation (i.e., quarantine mandated by local regulations)

### IX. Statistical Consideration and Analytical Plan

### (a) Study Hypothesis

The primary objective is to assess the safety and tolerability of the hUBC composition provided herein. No formal hypothesis is tested for the primary endpoint.

### (b) Analyzed Population

The analysis of primary safety endpoint will be based on safety population, the safety set (SS). The results for efficacy endpoint will be concluded mainly based on full analysis set (FAS) population. Analyzed populations are defined as below:
Safety set (SS)—all randomized patients who take at least one dose of IP.
Full analysis set (FAS)—all randomized patients who take at least one dose of IP and have at least one evaluable data for efficacy measurement.

### (c) Statistical Analysis

### General Considerations

All data are summarized by visit. For continuous variables, the number of observations (n), mean, median, standard deviation (SD), range (minimum and maximum), and 95% CI will be presented. For categorical variables, frequencies and proportions are provided.

### Demographic Analysis

All baseline characteristics of demographic data at study entry are listed by patients. Summary statistics is performed for each demographic variable. Demographic data will be summarized and tabulated. Continuous variables will be summarized using n, mean, SD, median, range (minimum and maximum), and 95% CI. Frequencies and percentages will be reported for all categorical data.

### Primary Endpoints Analysis

The primary endpoint is the incidence of TEAEs. Count and percentage of TEAEs/SAEs by severity/causality is provided.

TEAEs/AEs are to be coded using the Medical Dictionary for Drug Regulatory Affairs (MedDRA®, version 25.0 or later) and presented as summary tabulations, including categorical information of interest such as severity, causal relationship to IP, outcome, and action taken. AEs occur between screening and the time of IP administration will be considered as “pre-treatment” and will not be analyzed. All other AEs occur starting from IP administration will considered to be “treatment-emergent”. AEs will be listed by patient and will be summarized in terms of maximum severity and relationship to the hUCB composition.

Summary tables of AEs and SAEs occurring during the study will be generated. Incidence of each TEAE and proportions of severity of TEAEs will be tabulated. TEAEs resulting in discontinuation of hUCB treatment or withdrawal from the study, Grade 3 or higher, SAEs, and deaths are tabulated.

### Secondary Endpoint Analyses

Secondary endpoint for changes in fatigue score as measured by CFQ-11 will be analyzed. Continuous variables will be summarized using n, mean, SD, median, range (minimum and maximum), and 95% CI. For comparison of changes score between two groups, the Wilcoxon rank-sum test is to be used.

### Exploratory Endpoints Analyses

Exploratory endpoints for changes in
Severity of fatigue measured by PGI-S
FRAIL scale,
Life quality measured by EQ-5D-5L,
Cognitive function measured by MoCA, and
Biomarkers of cognitive impairment and fatigue will be analyzed.

Continuous variables will be summarized using n, mean, SD, median, range (minimum and maximum), and 95% CI. Frequencies and percentages will be reported for all categorical data. For comparison of continuous variables between two groups, the Wilcoxon rank-sum test will be used. For categorical variables, frequencies and proportions will be presented and Fisher's exact test is to be used.

### Other Safety Analyses

Pre-treatment and concomitant medication administered during the study will be listed. Concomitant medications will be coded using the WHO Drug Dictionary.

Results of vital signs, physical examinations, and laboratory tests will be listed. Individual continuous variable change from baseline in vital signs and laboratory values will be calculated and summarized descriptively. Frequencies and percentages will be reported for all categorical data.

### Results

Results obtained from five post-COVID patients, three being in the hUCB treatment group and two being in the placebo group, are summarized below.

(a) hUCB Composition Alleviates Fatigue in Post-COVID Patients

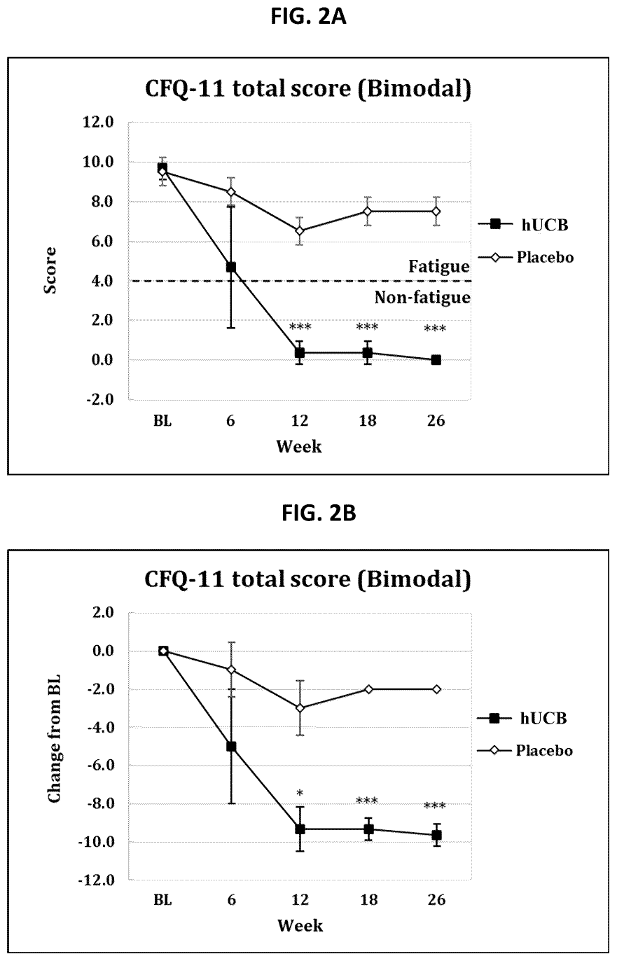

Post-COVID patients were treated with the hUCB composition disclosed above in the Phase IIa clinical trial described herein. The patients were assessed by CFQ-11 bimodal scoring to determine whether they suffer from fatigue (score ≥4). The hUCB group showed significantly lower scores than the placebo group and showed no fatigue at weeks 12, 18 and 26 after hUCB infusion. FIG. 2A. These results indicate that hUCB successfully alleviated fatigue in post-COVID patients. Comparing to the baseline, the hUCB group also showed a significantly greater decrease of bimodal scores than the placebo group at weeks 12, 18 and 26 after hUCB infusion.

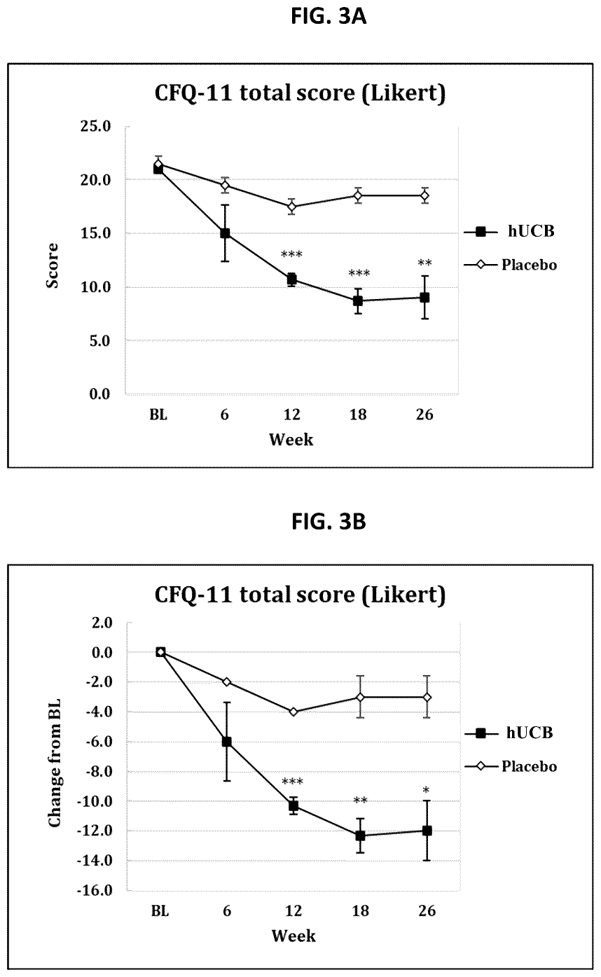

Further, the patients were assessed by CFQ-11 likert scoring to determine the severity of fatigue. The hUCB group showed significantly lower scores than the placebo group at weeks 12, 18 and 26 after hUCB infusion, indicating that the patients in the hUCB group showed a significant relief of fatigue. FIG. 3A. Comparing to baseline, the hUCB group also showed a significantly greater decrease of Likert score than the placebo group at weeks 12, 18 and 26 after hUCB infusion. FIG. 3B.

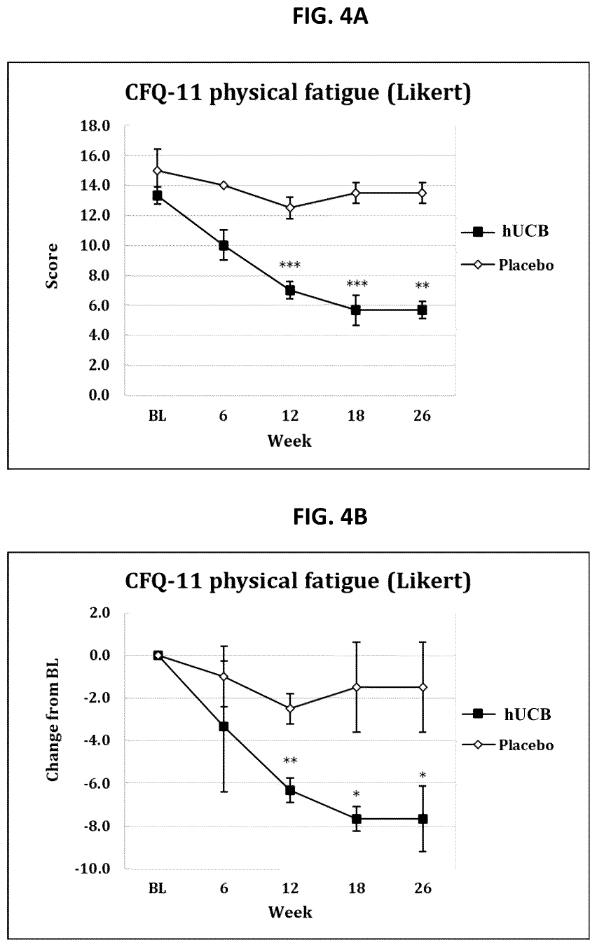

The patients were also assessed by CFQ-11 Likert scoring to determine the severity of physical fatigue. The hUCB group showed significantly lower scores than the placebo group at weeks 12, 18 and 26 after hUCB infusion, indicating that the patients in the hUCB group showed a significant relief of fatigue. FIG. 4A. Comparing to baseline, the hUCB group also showed a significantly greater decrease of Likert score than the placebo group at weeks 12, 18 and 26 after hUCB infusion. FIG. 4B.

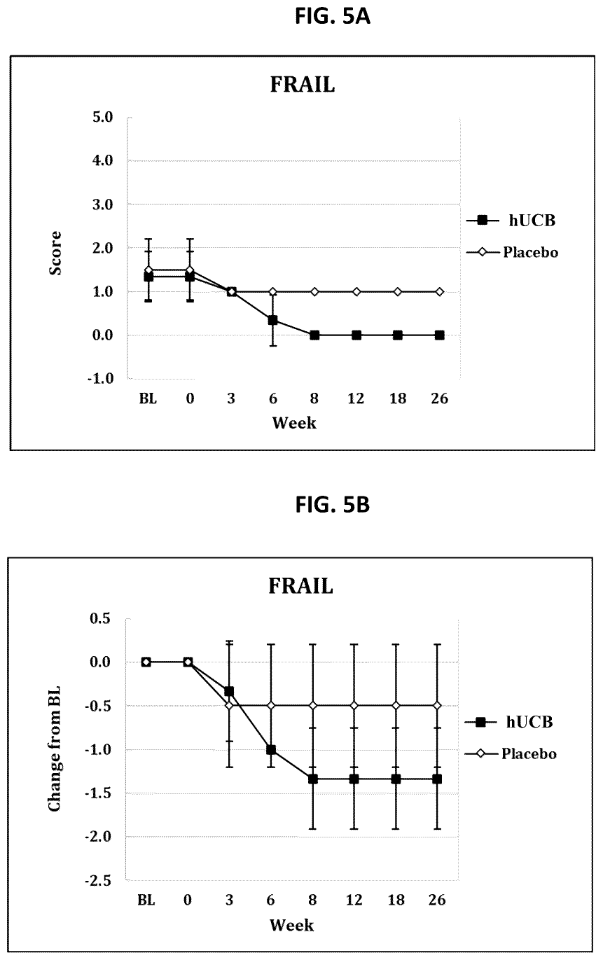

In addition, the patients were assessed by FRAIL questionnaire to determine changes in FRAIL scale after hUCB treatment. As shown in FIG. 5A, the hUCB group showed lower FRAIL scores starting at week 6 after hUCB treatment as compared with the placebo group, indicating that hUCB treatment led to healthier status. Similar results are observed when the FRAIL score changes are shown based on changes from baseline. FIG. 5B.

(b) Impact of hUCB Composition on Life Quality in Post-COVID Patients

Patients of both the hUCB group and placebo group were assessed by the EQ-5D-5L scores to determine impact of the hUCB treatment on mobility, self-care, usual activities, pain or discomfort, and anxiety or depression in post-COVID patients.

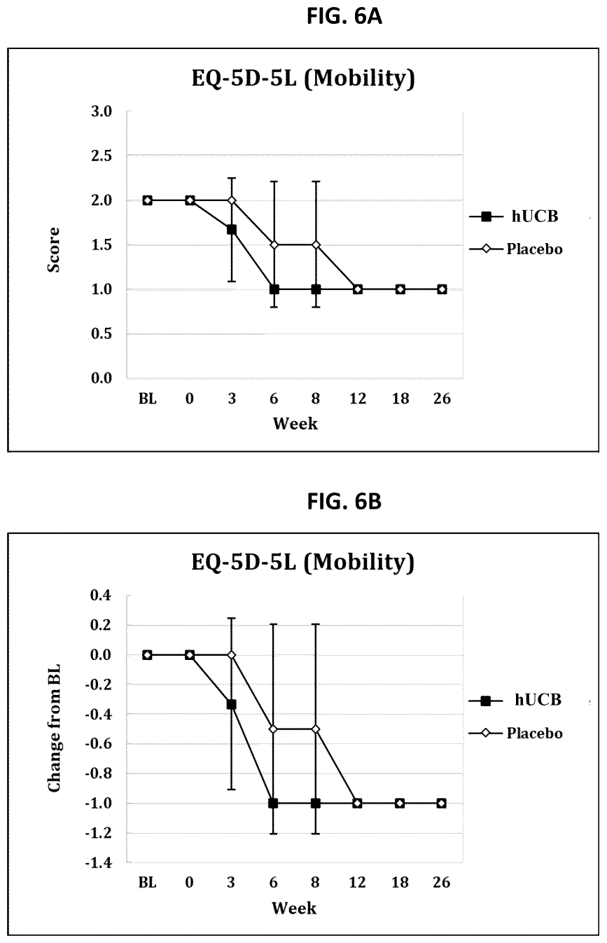

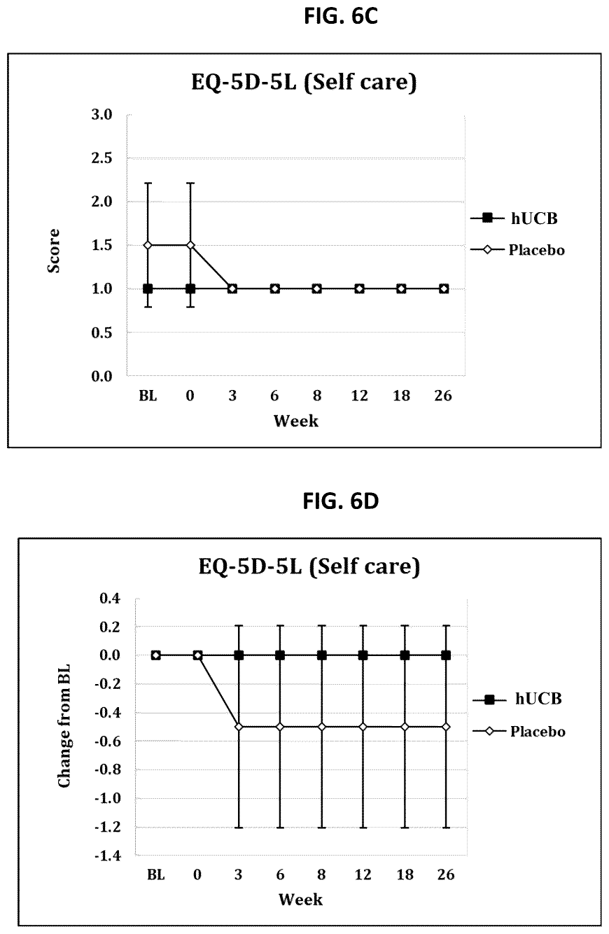

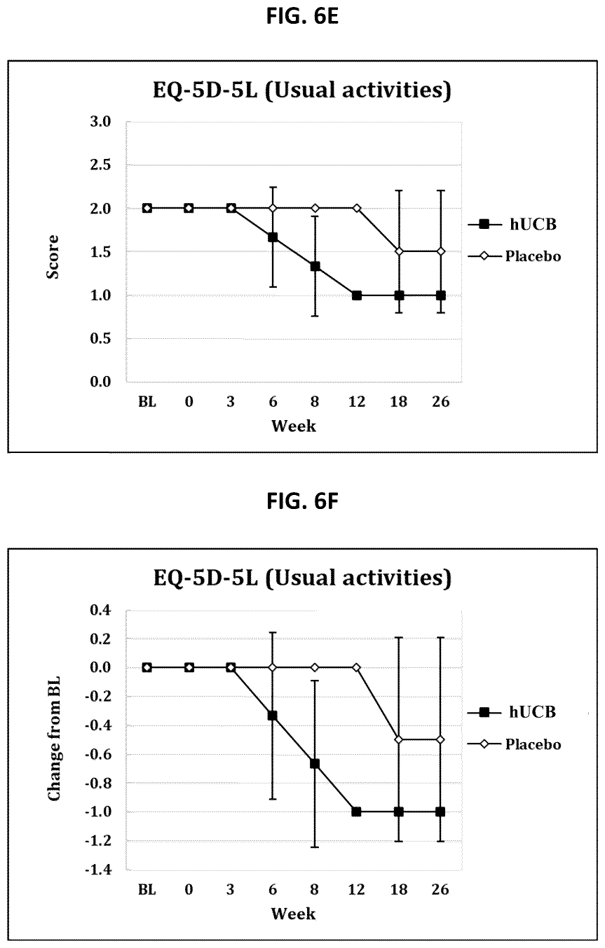

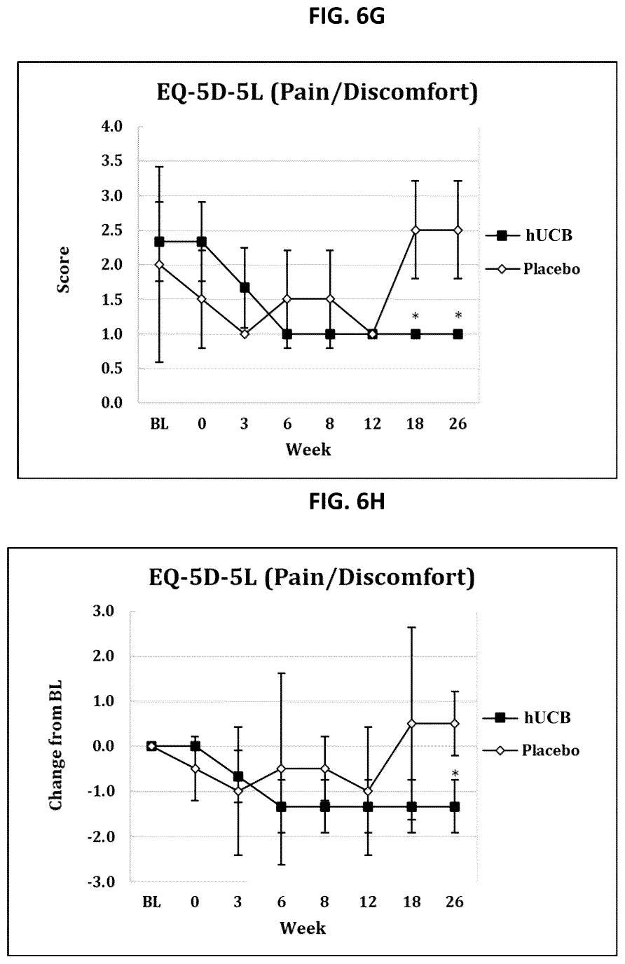

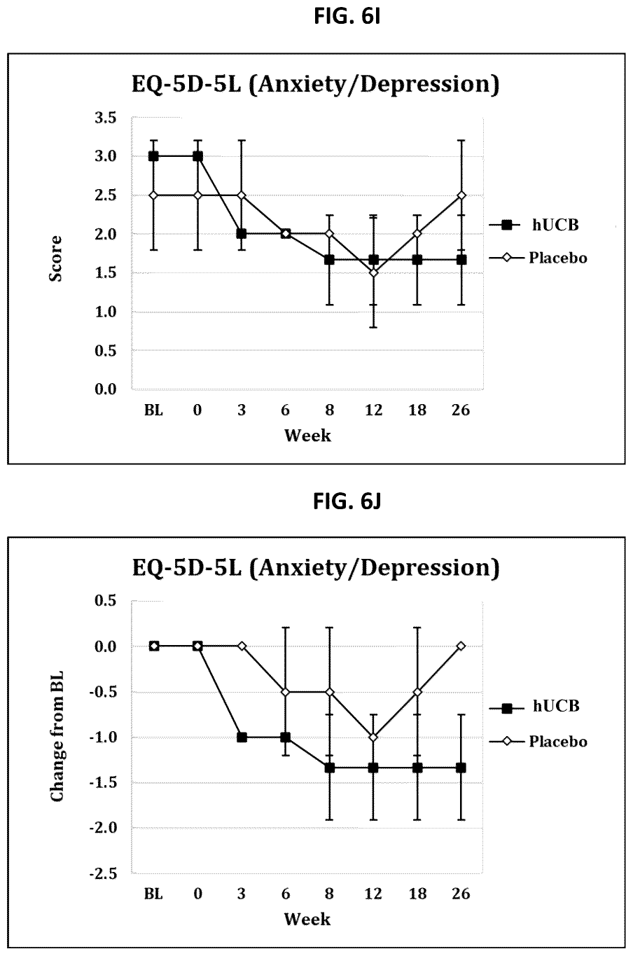

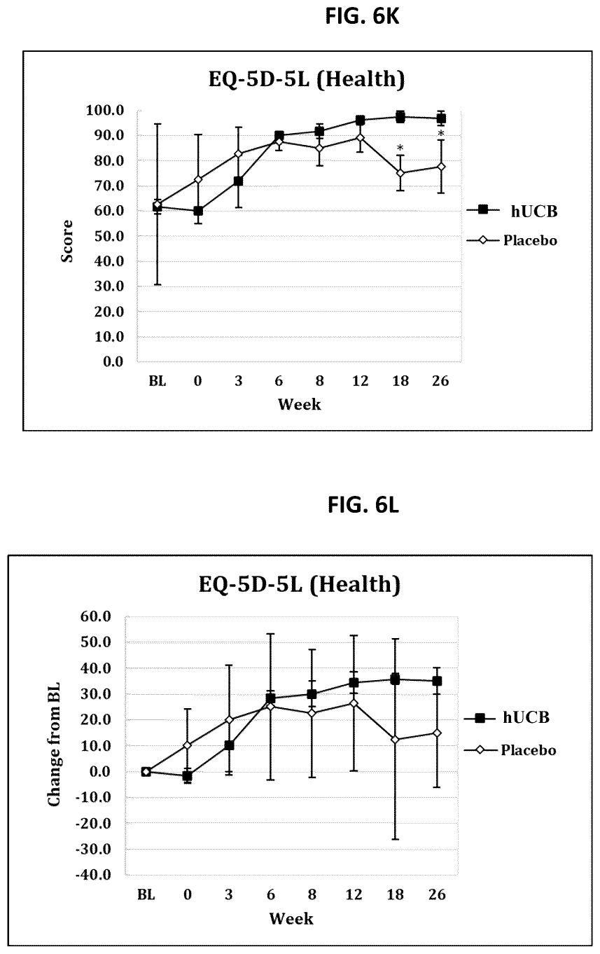

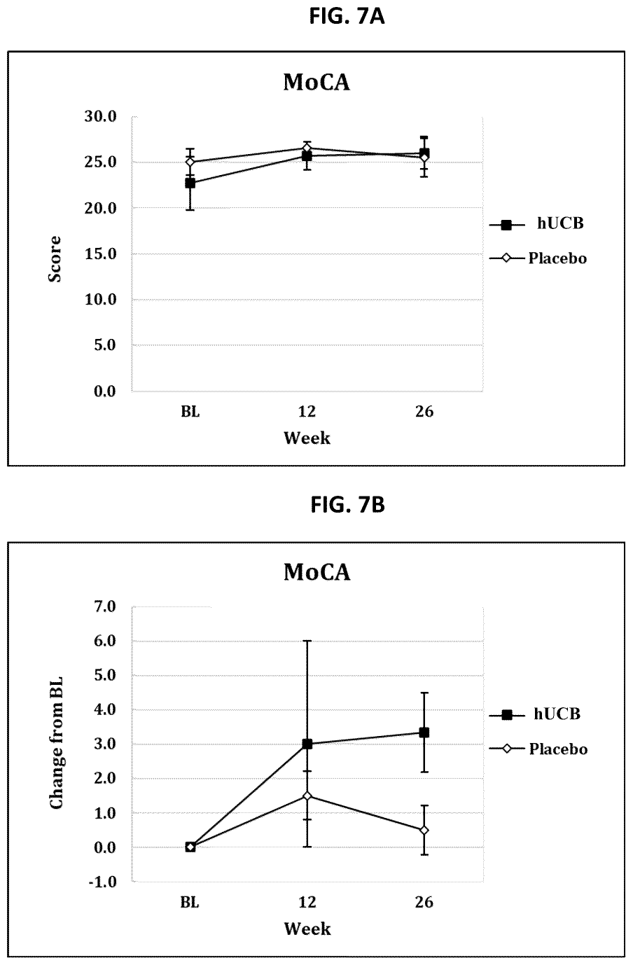

Changes of the EQ-5D-5L scores with respect to mobility, self-care, and usual activities in the hUCB group relative to the placebo group are shown in FIGS. 6A-6F. With respect to pain or discomfort, the hUCB group showed significantly lower scores than the placebo group at weeks 18 and 26 after infusion, indicating that hUCB significantly alleviated pain and discomfort in post-COVID patients as compared with the placebo control group. FIG. 7G. Comparing to baseline, the hUCB group also showed a significantly greater decrease of score than the placebo group at week 26 after infusion. FIG. 7H. FIGS. 7H-7K show EQ-5D-5L scores with respect to anxiety/depression and health in the hUCB group versus the placebo group after treatment. The results indicate benefits of hUCB treatments with respect to these features.

(c) Impact of hUCB on Cognitive Ability

Patients of both the hUCB group and placebo group were assessed by the MoCA scores to determine impact of the hUCB treatment on cognitive capacity of the post-COVID patients. As shown in FIGS. 7A and 7B, the hUCB group showed higher MoCA scores (specifically the changes from baseline) relative to the placebo group, indicating beneficial effects of hUCB on post-COVID patients with respect to cognition.

(d) Changes of Biomarker Levels in Post-COVID Patients Treated with hUCB

Levels of biomarkers associated with frailty, cognition, and/or fatigue, including Cystatin C, IGF-1, DHEA-S, were measured in patients treated with hUCB or the placebo.

Briefly, plasma samples were obtained from patients at pre-dosing, 6 weeks and 20 weeks after third dosing. Cystatin C was determined by latex enhanced immunotubidimetry assay (a measuring kit developed by Denka Seiken Co., Ltd.) on autoanalyzer; IGF-1 was determined by immunoradiometricassay (a measuring kit developed by Immunotech, BeckmanCoulter) on autoanalyzer; DHEA-S was determined by electrochemiluminescence immunoassay (a measuring kit developed by Roche Diagnostics GmbH) on autoanalyzer.

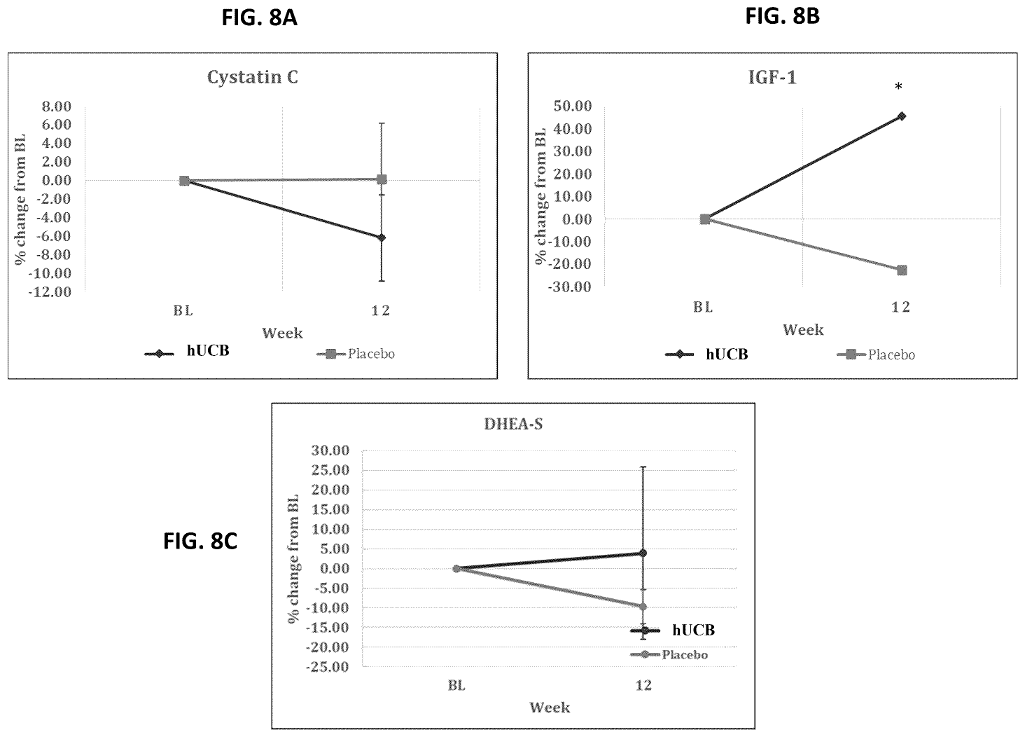

Cystatin C is reported to elevate with frailty and renal dysfunction, chronic inflammation, hypertension, and severity of COVID-19 with neurological symptoms. Cystatin C is also reported as a biomarker for post-stroke fatigue. Gunasekaran et al., Aging Med. 2018, 1(2): 149-153 and Yang et al., Brain Behav. 2021 11(2): e01969. As shown in FIG. 8A, hUCB treatment significantly reduced the level of Cystatin C (see comparison between the hUCB group and the placebo group).

The insulin-IGF-1 signaling pathway regulates energy metabolism and cell survival. IGF-1 is reported to decrease in frail older adults or in patients with cognitive impairment. Mohamed et al., Virology Journal, 2023, 20:94 and Angelini et al., Arch Gerontol Geriatr. 2009, 49 Suppl 1:5-12. The results from this study show that hUCB significantly increased the level of IGF-1 in patients receiving the treatment. FIG. 8B.

DHEA-S is reported as a biomarker associated with fatigue. The level of DHEA-S is lower in patients having chronic fatigue syndrome (CFS) and drops about 10-20% in each decade of lifespan. The average level of DHEA-S in CFS patients is 150.1±60.9 g/dL, while that in normal human subjects is 254.2±96.3 g/dL. As shown in FIG. 8C, hUCB significantly increased the level of DHEA-S in patients receiving the treatment.

(e) Changes of Inflammatory Cytokine Levels in Post-COVID Patients Treated with hUCB

Certain inflammatory cytokines, including CTACK, IP-10, IL-18, and NCAM-1, were reported to be elevated in COVID patients. Xu et al., Signal Transduction and Targeted Therapy 5:100 (2020); Davis et al. Nature Reviews Microbiology 21:133-146 (2023), and Satis et al., Cytokine, 2021, 137:155302. Further, inflammatory cytokines like KLK-6 and CHI3L1/YKL-40 were reported as being elevated in patients with neurological symptoms or cognitive deficits, either or without COVID-19. Sun et al., Cells, 2021, 10(2): 386 and Libreros et al., J. Leukoc. Biol., 2015, 98(6): 931-936.

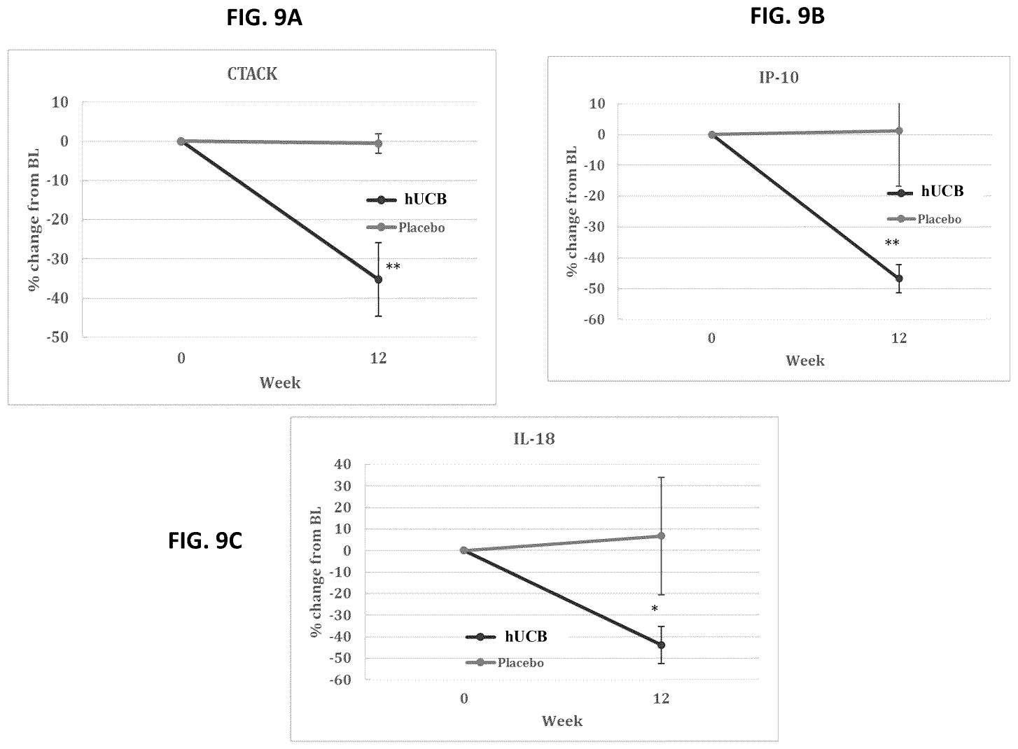

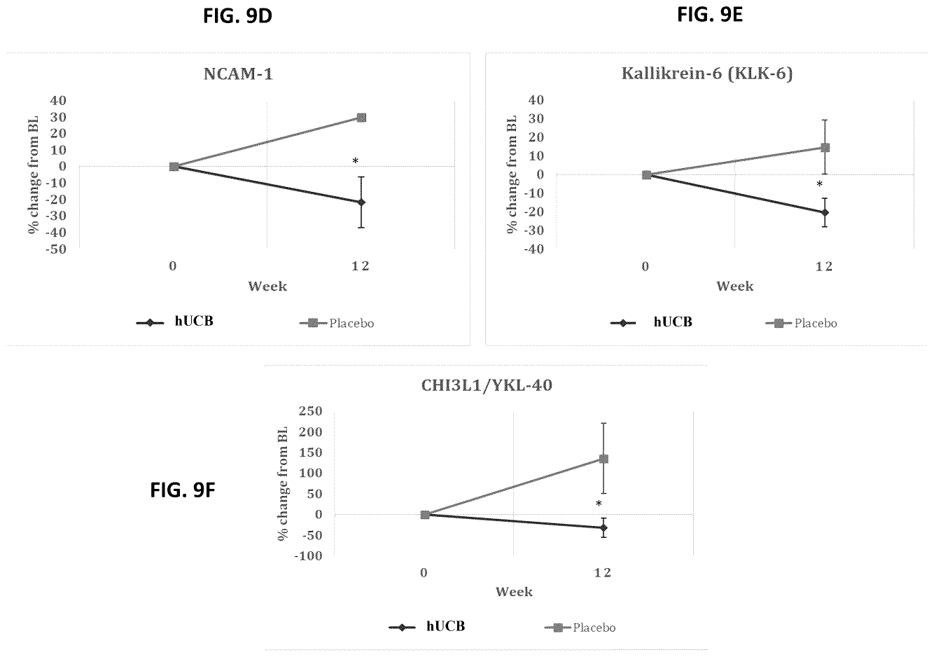

In this study, plasma samples were collected from patients treated with hUCB or placebo and analyzed for levels of inflammatory cytokines such as those noted above following routine practice. As shown in FIGS. 9A-9F, hUCB significantly reduced the levels of these inflammatory cytokine in COVID-19 patients.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the patient matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/of” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for alleviating fatigue, comprising administering to a subject in need thereof an effective amount of a human umbilical cord blood composition, which comprises hematopoietic progenitor cells, monocytes, lymphocytes, granulocytes, or a combination thereof.

2. The method of claim 1, wherein the subject is a human patient suffering from fatigue.

3. The method of claim 2, wherein the human patient has post-COVID syndrome or chronic fatigue syndrome.

4. The method of claim 1, wherein the human umbilical cord blood composition further comprises 5-10% dimethyl sulfoxide (DMSO) by volume and 0.5-1% dextran by volume.

5. The method of claim 1, wherein the human umbilical cord blood composition is cryopreserved prior to administration.

6. The method of claim 1, wherein the administering step comprises one or more doses of the human umbilical cord blood composition.

7. The method of claim 6, wherein each dose is about 1×10⁷ to about 9.0×10⁷ total nucleated cells, optionally about 1×10⁷ to about 8.6×10⁷ in the human umbilical cord blood composition per kilogram of the subject.

8. The method of claim 7, wherein each dose is about 1×10⁷ total nucleated cells in the human umbilical blood composition per kilogram of the subject.

9. The method of claim 7, wherein each dose of the human umbilical cord blood composition has a volume of 25 mL.

10. The method of claim 6, wherein the administering step comprises three doses of the human umbilical cord blood composition, each being about 1×10⁷ to about 9.0×10⁷ total nucleated cells per kilogram of the subject; and wherein two consecutive doses of the three doses are three weeks ±3 days apart.

11. The method of claim 10, wherein the administering step comprises three doses of the human umbilical cord blood composition, each being about 1×10⁷ total nucleated cells per kilogram of the subject; and wherein two consecutive doses of the three doses are three weeks ±3 days apart.

12. The method of claim 1, wherein the human umbilical cord blood composition is allogenic to the subject.

13. The method of claim 1, wherein the ABO/Rh type of blood cells in the human umbilical cord blood composition matches that of the subject.

14. The method of claim 1, wherein the subject has SARS-CoV-2 infection at least 6 months prior to the first dose of the human umbilical cord blood composition with post-COVID syndrome.

15. The method of claim 1, wherein the human umbilical cord blood composition is administered to the subject by intravenous infusion.

16. The method of claim 15, wherein the subject is an adult human patient and the infusion rate is 0.5-5 mL per minute, optionally 1.5-2 mL per minute.

17. The method of claim 1, further comprising monitoring occurrence of adverse effects after administration of the human umbilical cord blood composition and reducing dose or discontinuing treatment when a severe adverse effect occurs.

18. The method of claim 1, further comprising measuring:
(a) a level of one or more inflammatory cytokines in one or more blood samples of the subject, and/or
(b) a level of one or more biomarkers associated with frailty, cognition, and/or fatigue in the one or more blood samples;
wherein the blood samples are collected before, during, and/or after administration of the human umbilical blood cord composition.

19. The method of claim 18, wherein the inflammatory cytokines are selected from the group consisting of cutaneous T cell-attracting chemokine (CTACK), C-X-C motif chemokine ligand 10 (IP-10), interleukin 18 (IL-18), neural cell adhesion molecule 1 (NCAM-1), kallikrein-related peptidase 6 (KLK-6), and chitinase-3 like protein 1 (CHI3L1).

20. The method of claim 18, wherein the biomarker(s) associated with frailty, cognition, and/or fatigue comprises Cystatin C, insulin-like growth factor 1 (IGF-1), and/or dehydroepiandrosterone sulfate (DHEA-S).
